(19) United States
(12) Patent Application Publication
(10) Pub. No.: US 2006/0036259 A1
(43) Pub. Date: Feb. 16, 2006

(54) Spine treatment devices and methods

(76) Inventors: Allen L. Carl, Slingerlands, NY (US); Dan Sachs, Minneapolis, MN (US)

Correspondence Address: PETERS, VERNY, JONES & SCHMITT LLP, Suite 230, 425 Sherman Avenue, Palo Alto, CA, 94306, US

(21) Appl. No.: 11/197,037

(22) Filed: Aug. 3, 2005

Related U.S. Application Data
(60) Provisional application No. 60/598,882, filed on Aug. 3, 2004.

(51) Int. Cl. A61B 17/58

(52) U.S. Cl. 606/90

(57) ABSTRACT

A device and method of spine distraction is provided.

### RELATED APPLICATION DATA

The present application claims the priority of Provisional Application No. 60/598,882 filed Aug. 3, 2004 and entitled: Spine Treatment Devices and Methods.

### FIELD OF THE INVENTION

The invention relates to devices to treat the spine, including but not limited to spinal distraction devices and other spinal treatment devices.

### GENERAL BACKGROUND

Certain spine conditions, defects, deformities (e.g., scoliosis) as well as injuries may lead to structural instabilities, nerve or spinal cord damage, pain or other manifestations. Back pain (e.g., pain associated with the spinal column or mechanical back pain) may be caused by structural defects, by injuries or over the course of time from the aging process. For example, back pain is frequently caused by repetitive and/or high stress loads on or increased motion around certain boney or soft tissue structures. The natural course of aging leads to degeneration of the disc, loss of disc height, and instability of the spine among other structural manifestations at or around the spine. With disc degeneration, the posterior elements of the spine bear increased loads with disc height loss, and subsequently attempt to compensate with the formation of osteophytes and thickening of various stabilizing spinal ligaments. The facet joints may develop pain due to arthritic changes caused by increased loads. Furthermore, osteophytes in the neural foramina and thickening of spinal ligaments can lead to spinal stenosis, or impingement of nerve roots in the spinal canal or neural foramina. Scoliosis also creates disproportionate loading on various elements of the spine and may require correction, stabilization or fusion.

Spine surgeons have long treated pain from instability or arthritic changes of the spine with fusion. Fusion involves removal of the native disc, packing bone graft material into the resulting intervertebral space, and anterior stabilization, e.g., with intervertebral fusion cages or posterior stabilization, e.g., supporting the spinal column with internal fixation devices such as rods and screws. Laminectomies and related procedures have been performed to treat spinal stenosis pain or from impingement of nerve roots in the neural foramina. Such procedures involve removing remove bone, calcifications or other growth that closes around or impinges on spinal nerves, sac centrally, and nerve roots. Sometimes these procedures include reinforcement of the posterior spine with rod and screw fixation.

More recently, as an alternative to laminectomies and related procedures, implants have been proposed that distract the spine from a posterior approach. In particular, a wedge-like implant inserted between two adjacent spinous processes has been proposed to relieve pressure on spinal nerves and nerve roots. A kyphosis is induced, which opens the space of the spinal canal and neural foramen, thereby reducing the effect of spinal stenosis. However, this type of distraction of adjacent spinous processes is suboptimal for several reasons: The resulting kyphosis is non-physiologic, leading to increased load on the anterior portion of the disc and the vertebral bodies. This can increase the risk of disc degeneration and vertebral compression fracture. The implant tends to bend the spine forward. Bone may collapse around the spinous process. The implant may weaken, tear, or stretch stabilizing ligaments of the spine, such as the supraspinous ligament, interspinous ligament, ligamentum flavum, posterior longitudinal ligament, or capsule of the zygapophyseal joint. The amount of distraction is not adjustable to the specific amount of stenosis, and cannot be easily readjusted months to years after the device has been implanted.

It would accordingly be desirable to provide a distraction device that reduces or avoids some or all of these issues.

The typical techniques for fusion, distraction, decompression, and dynamic stabilization require open surgical procedures with removal of stabilizing muscles from the spinal column, leading to pain, blood loss, and prolonged recovery periods after surgery due in part to the disruption of associated body structures or tissue during the procedures. Accordingly, it would be desirable to provide less invasive devices and methods for treating pain or discomfort associated with the spinal column. It would also be desirable to provide such devices and methods that are less damaging to associated tissue.

Some less invasive or “less disruptive” procedures have been proposed to posteriorly or laterally access the spine and create spaces adjacent the spine for posterior stabilization procedures. Typically these less disruptive procedures involve creating spaces between adjacent portions (e.g. between pedicles) so that stabilizing devices can be positioned between the portions and attached, e.g. to the pedicles. However, these stabilization devices typically involve the use of 4 pedicle screws (each having a risk associated with it when placed in the spine), two on each side of a motion segment, and are not ideally suited for percutaneous stabilization required across more than one or two segments. Accordingly, it would be desirable to provide a less invasive or less disruptive segmental spine stabilization procedure and implant that has a reduced risk of damage or injury. It would also be desirable to provide a minimally invasively implanted posterior spine system that may be used to stabilize more than two motion segments.

Spine surgeons commonly use metallic or polymeric implants to effect or augment the biomechanics of the spine. The implants frequently are attached or anchored to bone of the spine. Sites typically considered appropriate for boney attachment have high density or surface area, such as, for example, the pedicle bone, the vertebral body or the cortical bone of the lamina. The spinous process contains thin walls of cortical bone, and thus, has been considered as not ideal for anchoring spinal implants as they may not support the implants under physiologic loads, or the intermittent high loads seen in traumatic situations. Fixation has been attempted from spinous process to spinous process with poor results.

A translaminar facet screw as used by some surgeons goes through the base of spinous process to access the cancellous bone of the lamina. A disadvantage of this device is that it is not suitable for attaching to a pedicle screw and the depth and angle during deployment can be very difficult to track or visualize, thus increasing the possibility that the screw would extend into the spinal canal. A facet screw is screwed between opposing facets of a zygapophyseal joint.

### SUMMARY

One aspect present invention is directed to providing a device and method for alleviating discomfort and or deformity associated with the spinal column. Another aspect of the present invention is directed to providing a minimally invasive implant and method for alleviating discomfort associated with the spinal column. Another aspect of the present invention provides an anchoring device and method that requires less surrounding tissue damage or disruption. Another aspect of the present invention provides reinforcement of the spinous process for use in various spinal systems. Another aspect of the invention provides a minimally invasive, non-invasive, or remote adjustment or lengthening of an orthopedic device. Another aspect of the invention provides a minimally invasive, non-invasive, or remote adjustment or lengthening of a stabilization or distraction device. Another aspect of the present invention also provides an implant system and device suitable for minimally invasive, minimally disruptive and/or percutaneous posterior deployment across a plurality of motion segments and more than two motion segments. Different aspects of the invention may provide distraction forces to relieve pressure on certain structures, compression forces to fix or stabilize motion across structures, shock absorbing qualities to help relieve load from certain structures, and therapeutic activity to reduce inflammation and pain. Other aspects of the invention may supplement or bear load for degenerated, painful, or surgically removed joints, e.g., the facet joint. Another aspect of the invention may provide a method and system for treating deformities such as scoliosis. Other aspects of the invention may include sensors associated with implants or implanted at or near the bones, soft tissue, or joints of the spine and may provide feedback regarding the joint on an ongoing basis. The sensors may also be part a feedback system that alters a property of an implant in response to sensing information. Another aspect of the invention may provide a device or method for delivering therapeutic substances at or near the spine.

In accordance with one aspect of the invention, a reinforcement structure is provided for supporting the spinous process and if desired, in addition, the lamina of a spine, e.g., for securing portions of the devices to the spine. The invention further provides a method and system for forming or implanting such structure in the spinous process or a region of cancellous bone in the lamina of a spine. The reinforcement system may include one or more systems of reinforcement and may be used before, during and/or after a spinal device (e.g. a stabilization, distraction or prosthetic device, etc.) is coupled to the spinous process.

Various aspects of the invention are set forth in the description and/or claims herein.

### BRIEF DESCRIPTION OF THE DRAWINGS

FIG. 1A is a lateral posterior view of a vertebra with a reinforcement structure in accordance with the invention.

FIG. 1B is a side view of the vertebra and reinforcement structure of FIG. 1A.

FIG. 2A is a lateral posterior view of a vertebra with a reinforcement structure in accordance with the invention.

FIG. 2B is a side view of the vertebra and reinforcement structure of FIG. 2B.

FIG. 3A is a lateral posterior view of a vertebra with a reinforcement structure in accordance with the invention.

FIG. 3B is a side view of the vertebra and reinforcement structure of FIG. 3A.

FIG. 4A is a lateral posterior view of vertebrae with a reinforcement structure and implant in accordance with the invention.

FIG. 4B is a side view of the reinforcement structure and implant of FIG. 4A.

FIG. 4C is a top view of a reinforcement structure and implant in accordance with the invention.

FIG. 4D is a posterior view of the reinforcement structure and implant of FIG. 4C.

FIG. 5 is a posterior view of a reinforcement structure and implant in accordance with the invention.

FIG. 6 is a posterior view of a reinforcement structure and implant in accordance with the invention

FIG. 7 is a posterior lateral perspective view of an implant implanted in accordance with the invention.

FIG. 8 is a posterior lateral perspective view of an implant implanted in accordance with the invention.

FIG. 9A is a side schematic view of a distraction element in a first position in accordance with the invention.

FIG. 9B is a side schematic view of the distraction element of FIG. 9A in a second position in accordance with the invention.

FIG. 9C is a side schematic view of a distraction element in a first position in accordance with the invention.

FIG. 9D is a side schematic view of the distraction element of FIG. 9C in a second position in accordance with the invention.

FIG. 9E is a side schematic view of a distraction element in accordance with the invention.

FIG. 9F is a side schematic view of a distraction element in accordance with the invention.

FIG. 10A is a top view of a dynamic implant in accordance with the invention.

FIG. 10B is a posterior view of the implant as shown in FIG. 10A.

FIG. 11 is a schematic posterior portal cross sectional view of a reinforcement device and implant in accordance with the invention.

FIG. 12 is schematic posterior partial cross sectional view of a reinforcement device and implant in accordance with the invention.

FIG. 13A is an exploded perspective view of a reinforcement device and implant in accordance with the invention.

FIG. 13B is a top view of the reinforcement device and implant of FIG. 13A.

FIG. 14A is a schematic partial cross sectional view of an implant in accordance with the invention in a first position.

FIG. 14B is a schematic partial cross sectional view of the implant of FIG. 14A in a second, and implanted position.

FIG. 15A is a schematic partial cross sectional view of an implant in accordance with the invention in a first position.

FIG. 15B is a schematic partial cross sectional view of the implant of FIG. 15A in a second position.

FIG. 16A is a posterior lateral perspective view of an implant adjacent a removed joint segment in accordance with the invention.

FIG. 16B is a posterior view of the implant implanted as shown in FIG. 16A.

FIG. 17A is a posterior lateral perspective view of a distraction system implanted in a spine in accordance with the invention.

FIG. 17B is a side perspective view of the distraction system implanted in a spine as shown in FIG. 17A.

FIG. 17C is a top view of the distraction system implanted in a spine as shown in FIG. 17A.

FIG. 17D is a posterior perspective view of the distraction system implanted in a spine as shown in FIG. 17A.

FIG. 18 is a posterior lateral perspective view of an implant implanted in accordance with the invention.

FIG. 19 is a posterior lateral perspective view of an implant in accordance with the invention.

FIG. 20A is a posterior lateral view of a distraction system in accordance with the invention.

FIGS. 20B-20I are a schematic illustration of a method of implanting the distraction system of FIG. 20A.

FIG. 21 is a posterior lateral view of a distraction system in accordance with the invention.

FIG. 22 is a schematic side view of a connector of an implant in accordance with the invention.

FIG. 23 is a schematic side view of a connector of an implant in accordance with the invention.

FIG. 24 is a schematic perspective view of a connector in accordance with the invention.

FIG. 25 is a schematic side perspective view of a dynamic element in accordance with the invention.

FIG. 26 is a schematic side perspective view of an adjustable implant element in accordance with the invention.

FIG. 27 is a schematic side perspective view of an adjustable implant element in accordance with the invention.

FIG. 28 is a schematic side perspective view of an adjustable implant element in accordance with the invention.

FIG. 29 is a schematic posterior lateral perspective view of a therapeutic substance delivery device in accordance with the invention.

FIG. 30 is a schematic posterior lateral perspective view of a therapeutic substance delivery device in accordance with the invention.

### DETAILED DESCRIPTION

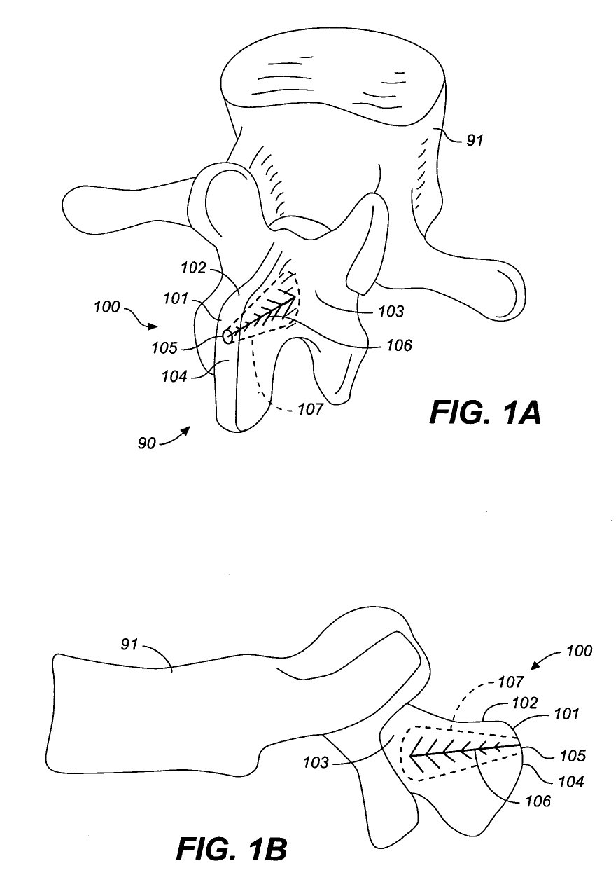

FIGS. 1A and 1B illustrate a reinforced posterior arch 100 of a first vertebra 91 of a spine 90, including a spinous process 101 and lamina 103. The first vertebra 100 of the spine 90 as illustrated includes a first spinous process 101 with a superior portion 102 having a posterior ridge 104 into which a hole 105 is drilled. The hole 105 may be drilled with a drill, a trocar, a large bore IV needle or similar sharp object through the external and relatively hard cortical bone, to reach the internal cancellous bone within the spinous process 101 and adjacent the lamina 103.

Once the cancellous bone is accessed, optionally, a tool such as a balloon tamp, or other expandable member or small crushing or drilling member is used to create a cavity 107 or cavities within the cancellous bone by compressing, crushing or drilling out the bone material. X-rays may be used to determine how far to drill into the bone. The cavity 107 may be in the spinous process, through to the base of the spinous process, or through the spinous process and into the lamina. In one embodiment the cavity is cone shaped or widens as it moves anteriorly towards the lamina.

A reinforcing material is then delivered into the cancellous bone or cavity 107 of the spinous process 101 and/or within the lamina 103. The material is selected to provide reinforcing properties to the spinous process 101 and/or lamina 103 sufficient to support (whether alone or in combination with other support elements) a spine support structure, a prosthesis, or other device attached to the spinous process and or supported lamina. The material may be a bone cement or polymer with strength and hardness properties selected to provide sufficient reinforcement to the region so that the spinous process may be used at least in part, to support an implant structure for attaching to and manipulating the biomechanics of the spine. Examples include but are not limited to polymers such as acrylic cement developed for use in vertebroplasty procedures. The material may be a flowable polymer material that cures within the cavity. Suitable materials may be readily selected by one of ordinary skill in the art.

Reinforcement structures may be placed within the cavity prior to, during or after injection of flowable material for further strength properties. As illustrated, an additional support structure 106 is provided within the cavity. The support structure 106 may be inserted through a cannula and released to expand as a spring-like or self-expanding member, into the cavity. The support structure 106 provides further support of the spinous process and/or lamina. Alternatively, or additionally, one or more posts or struts may be provided within the cavity or extending out of the spinous process or lamina from the area of cancellous bone, to supplement the support of the spinous process or lamina in combination with the polymer or other curable material. The reinforcement structures may be formed of a number of different materials such as, e.g., a metal or biocompatible polymer. Such reinforcement structures may also be used in other bony areas of the spine including the vertebra, the pedicles, facets, the transverse process, etc.

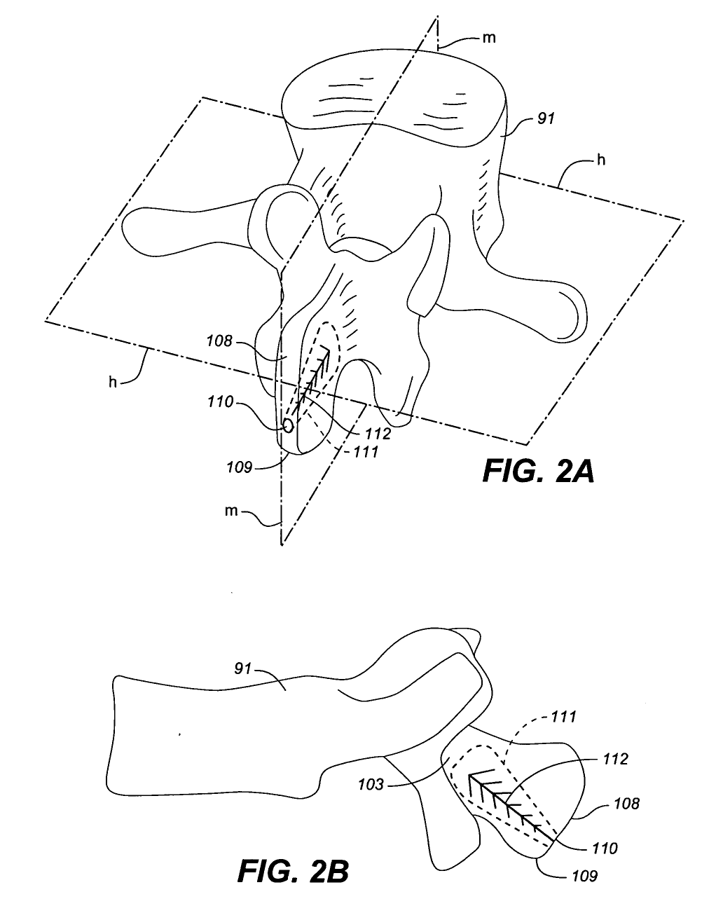

As shown in FIGS. 2A an 2B, an inferior portion 109 of a spinous process 108 may also be reinforced. Similarly a hole 110 is drilled in the inferior portion of the spinous process 108 and a cavity 111 is formed. The cavity 111 is similarly filled with a curable polymer and is reinforced by reinforcing elements 112 positioned within the cavity.

The reinforcement structure may be used in a number of applications including increasing the strength of healthy bone to support the load and fixation of orthopedic implants, as well as increasing the strength of bone weakened by osteoporosis, chronic steroid use, avascular necrosis, weakened by injury and cancer involving the bone. According to one aspect, the reinforcement structure comprises a material that provides sufficient strength including but not limited to suitable polymers, e.g. PEAK, titanium, steel and carbon fiber.

The stabilizing and/or distracting devices described herein may be formed of a material that provides sufficient column strength including but not limited to suitable polymers, e.g. PEAK, titanium, steel, and carbon fiber.

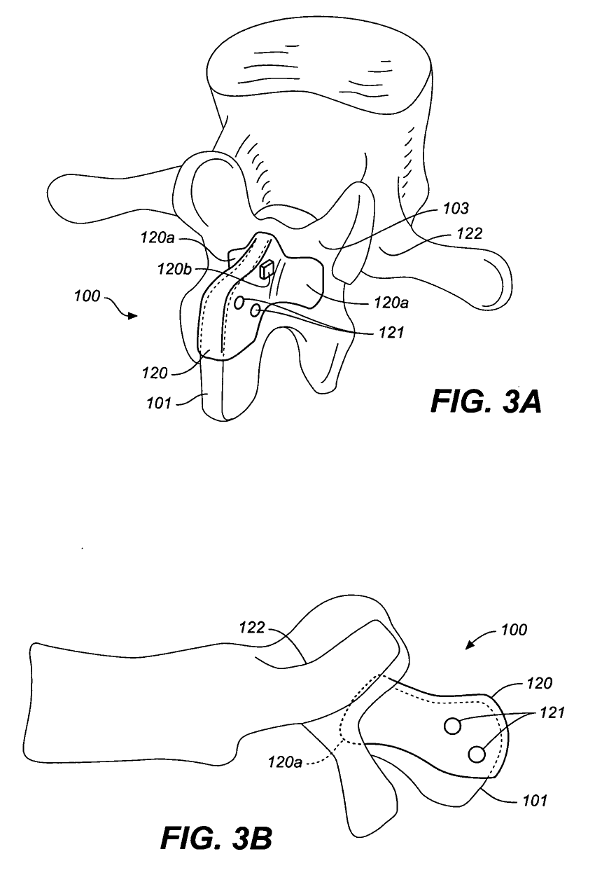

Referring to FIGS. 3A and 3B, an alternative support structure 120 is illustrated. The support structure 120 allows the anchoring of implants under physiologic loads on the spinous process 101 while shielding underlying bone from loads that would normally cause the bone to fracture. (The implants may alternatively or in addition be anchored or attached to the lamina 103, e.g., with addition of small screws, barbs or adhesive that engage with the lamina while avoiding injuring the spinal cord surrounded by the lamina.) The support structure 120 comprises a hood like element positioned over the posterior arch 100, i.e., the spinous process 101 and lamina 103 of a spine 90. The support structure 120 may be made of a moldable or malleable material (e.g. putty, formable ceramic, clay-like material, or a moldable polymer or malleable alloy or metal) that cures into or forms a solid, strong structure. Heat, light, catalysts, precursors, or local pressure and force, for example, may be used to make the hood moldable or firm. The support structure of filling material to support the spinous process may be constructed or formed of moldable composites that can cure into hard material such as, e.g., ground glass powder or glass fiber fillers mixed into an acrylic matrix and activated with light or other biophysical modalities. Other cements or other curable materials may be suitable as well. The support structure 120 further comprises openings 121 to guide drill bits and/or for the placement of screws, reinforcement posts, or other instruments or supplemental support structures. The support structure 120 may be anchored on the posterior arch by mold bending or forming the structure about the anatomy. The support structure 120 may be anchored into the lamina or spinous process by anchoring elements, such as, e.g., screws or barbs. The support structure 120 may also be anchored via screws or posts. Alternatively, the support structure 120 could be a preformed implant with contours that fit the anatomy of the posterior arch 100 or that are malleable or moldable to the anatomy. Also, the support structure 20 may be anchored into the pedicles 122 with screws, into the underlying bone with barbs, screws, bone anchors, or adhesives, over the edges of structures with hooks, or may be constructed of a plurality of pieces that may be assembled into one piece around the bone. Wings 120a of support structure may be placed over the lamina to spread the force of any device attached to the support structure 120.

As illustrated in FIGS. 3A and 3B, a sensor 120b, is positioned on the support structure 120. The sensor 120b may be embedded in the material. The sensor may sense stress on the support structure 120 from implants secured to it, or may sense other information that may be desirable to monitor. The sensor may include a communication element configured to communicate sensed information to an external device, e.g., when interrogated.

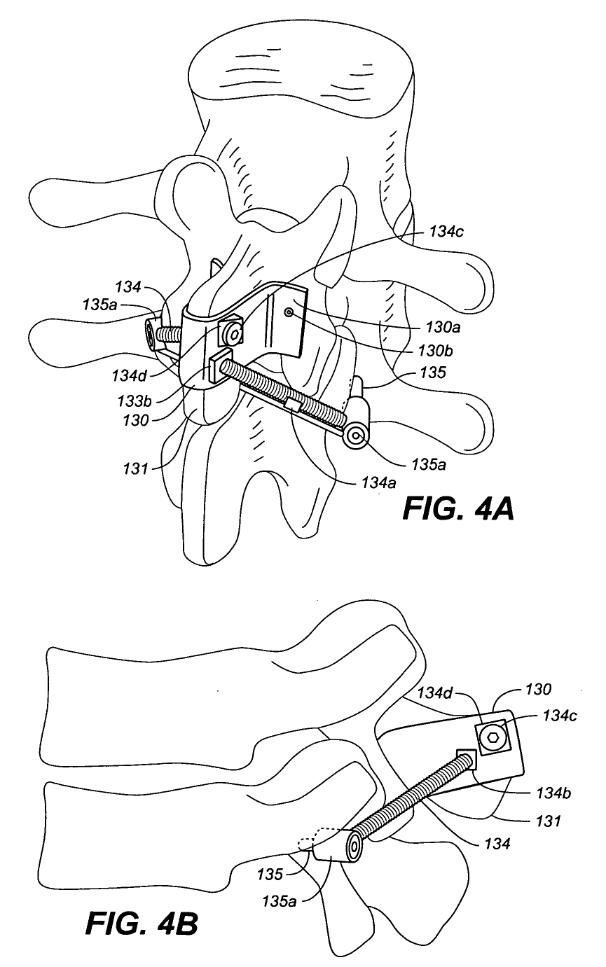

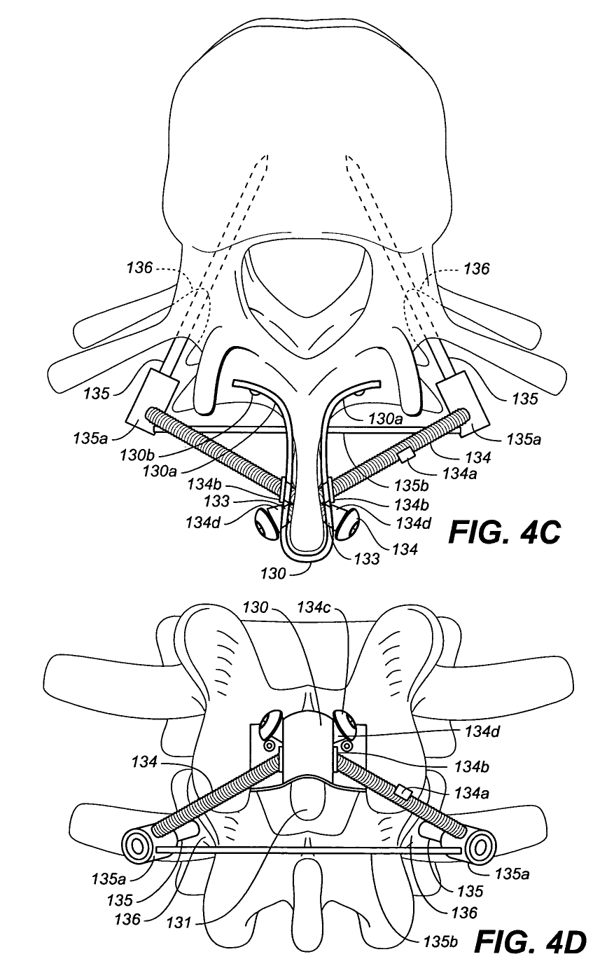

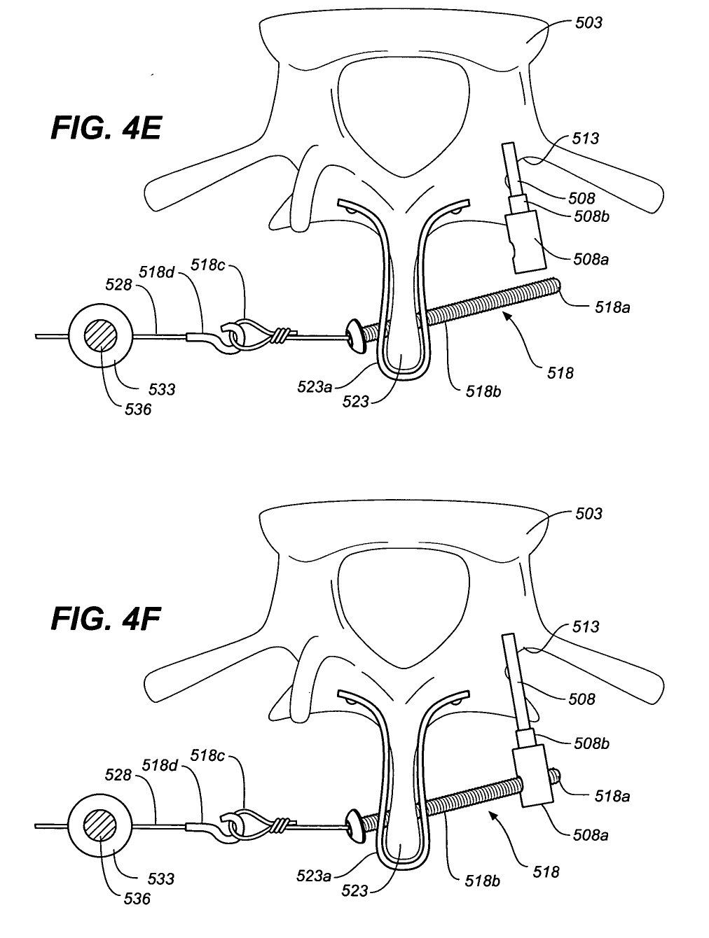

Referring to FIGS. 4A-4D, a support structure 130 is illustrated positioned over a posterior portion 132 of a spinous process 131 with wings 130a over the lamina 103 including small screws 130b into lamina 103. Wings 130a may help spread the force from any devices attached or coupled to the support structure 130. Pedicle screws 135 are anchored into pedicles 136 and are further anchored into the spinous process 131 through screws 134 positioned through holes 133 in the support structure 130. As shown in FIG. 4C, the screw 134 includes a sensor 134a that may be used to sense loads on the device. Use of such sensors is described further herein. The pedicle screw 135 includes a screw capture device 135a for receiving a screw or rod of a spinous process screw or other rod. The capture device 135a may be a polyaxial head of a pedicle screw it may include a hole, a threaded screw hole with a washer or cap. Cross bar 135b is positioned across the spine between heads of pedicle screws 135 to prevent pedical screws from creeping laterally. A wedge shaped nut 134d between the head 134c of the screw 134 and the support structure. Another nut 134b may be positioned between support structure 120 and pedicle screw, and secure against the support structure 120. These features may be used in a similar manner in the embodiments described herein.

The pedicle screw 135 may be configured to telescope outwards or inwards to be positioned to receive the screw head or rod of a spine device in a manner similar to that shown in FIGS. 4E and 4F. Referring to FIGS. 4E and 4F, a pedicle screw 508 is configured to telescope outwards or inwards to be positioned to receive the screw head or rod of a spinous process screw 518. The spinous process screw 518 is shown in FIG. 4E where, given the trajectory of the spinous process screw 518, its end does not intercept the capture device 508a of the pedicle screw 508. As shown in FIG. 4F the pedicle screw's trunk 508b is lengthened with a telescoping or other similar lengthening mechanism so that the end of the spinous process screw 518 may be positioned in the capture device 508a.

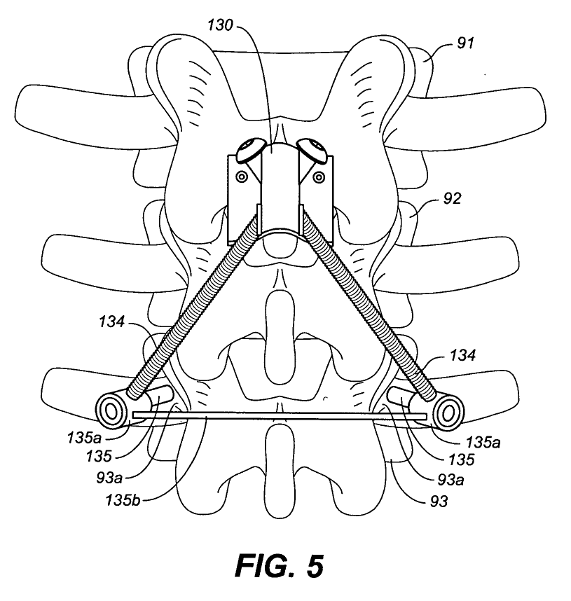

FIG. 5 illustrates the spinous process screws 134 coupled to a spinous process 101 of a first vertebra 91 through a hood or support structure 130 in a manner similar to that described above with respect to FIGS. 4A-4D. The screws 134 extend bilaterally across the posterior of a second vertebra 92 and are anchored to capture elements 135a of pedicle screws 135 anchored into pedicles 93a of a third vertebra 93.

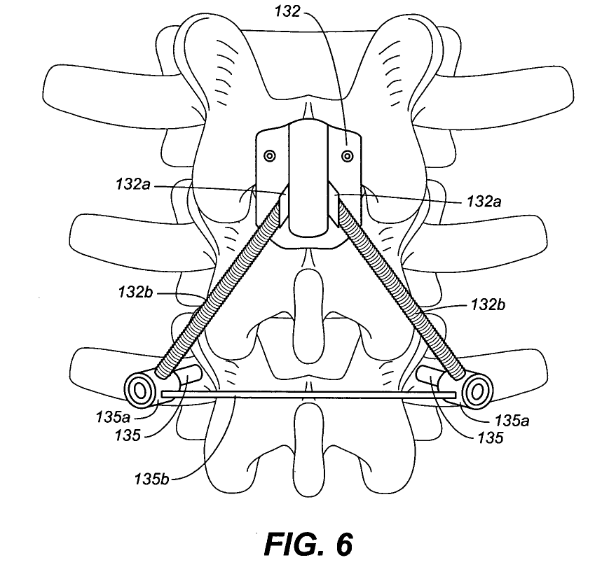

FIG. 6 illustrates a device for stabilizing or distracting the spine with pedicle screws 135 and cross bar 135b positioned as in FIG. 4D. Hood structure 132 includes openings for receiving screws 132b coupled to the hood 132 on one end and to the heads 135a of pedicle screws 135 and on the other end. The screws 132b do not penetrate the spinous process. Obliquely threaded nuts secure the screws 132b against the hood 132.

The reinforcement or supporting devices described herein may be used in conjunction with a number of different spine devices, including, for example, the various distraction, fusing or dynamic stabilizing devices described herein. The hoods or reinforcement devices herein may also be customized, for example by using stereolithography. The hoods or reinforcement devices may be used for example with a brace.

The devices described herein may be coupled to the spinous process using minimally invasive techniques. These techniques may include percutaneously accessing the spinous process and/or using dilators to access the spinous process at an oblique angle with respect to median plane m and/or horizontal plane h through the spine of the patient. An oblique skin stab wound is made to navigate to the spinous process, which may be exposed under direct vision. The spinous process screw or other distraction device is then screwed or positioned through the spinous process across or through the facet joint, and into a pedicle screw or attachment device stabilizing the facet joint. A similar screw may also be placed from the spinous process to the contralateral pedicle. The spinous process may be reinforced prior to or after placing the screw or other distraction device.

One aspect of the present invention provides a distraction device that distracts the joint in an upward or in less of a forward bending manner diminishing kyphosis formation. A distraction device in accordance with the invention lessens spinal stenosis and reduces stress on the facet joints. In accordance with one aspect of the invention, narrowing or stenosis of the neural foramen may be treated using a device configured to distract the facet joint.

In accordance with one aspect of the invention, a distraction system is provided where the system is anchored on opposite sides of a motion segment that would benefit from distraction. According to an embodiment, on opposite lateral sides of the motion segment, an expandable rod, screw, or other columnar support structure is attached. The length of the support structure may be adjusted to determine the degree or amount of distraction. Additionally, a spring or shock-absorbing element may be included in the distraction device. In accordance with one aspect of the invention, such distraction device may provided with screws 134 as illustrated in FIGS. 4A and 4D.

One aspect of the invention contemplates use of orthopedic implants that can be remotely lengthened after surgery, as needed. For example, the gait of patients after hip replacement surgery may be effected if the leg length of one limb is longer or shorter than the other. This invention would allow doctors to change the implant's length over time as needed to help restore normal gait. Other indications include surgical procedures where an external fixator is used in long bone fractures. According to the invention a distractor as described may be affixed at opposite ends, to opposite sides of other structures of the body, including, for example a hip joint. The distractor may be remotely actuated or less invasively accessed for distraction adjustments, including, e.g., post operatively, over the life of the prosthetic implant, or over time.

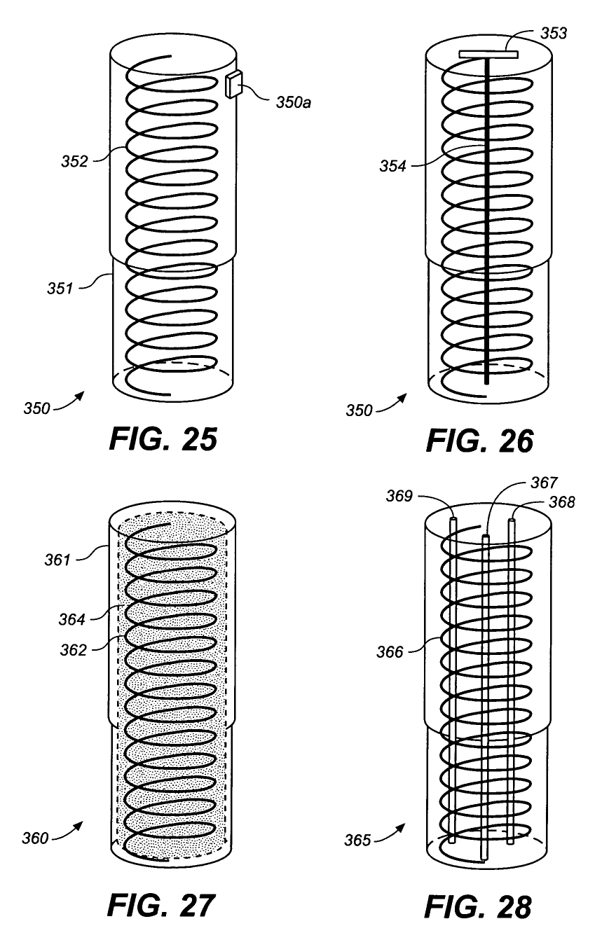

A variety of distraction systems are contemplated for distracting the adjacent vertebrae (including but not limited to the distractions systems disclosed herein), e.g., an expandable screw or rod or plate, telescoping implant, a distraction jack, an inflatable column, a column that lengthens when exposed to heat, fluids, ultrasound, or other biological, physical, or chemical catalysts (using, for example, a device constructed of a shape memory alloy or rheostatic fluids). The amount of distraction may be controlled remotely, by radiofrequency, electromagnetic energy, electrical, heat, ultrasound, and other means. The distracting member for example may comprise a remotely actuated realignment device or solenoid. The distraction may also be adjusted percutaneously or remotely according to one of these variations. The adjustments may be made over time, particularly if the disease progresses or other anatomical changes occur. This would allow adjustment of the amount of distraction as needed to a patient's symptoms long after surgery. The distraction adjustment may also be done with patient feedback. The distraction devices may also include a variety of different types of sensors that sense changing loads on the spine or on the device. For example, the distraction device may include a pressure sensor or a strain gauge. As noted above, the distraction device with spring properties may include a freeze or lock (for example, as described with respect to FIGS. 25-28 herein) that permits the device to be immobilized should a fusion type procedure be necessary to immobilize a patient's spine, for example at a later date with further wear or progression of disease. The flexibility or stiffness of the device may also be incrementally or progressively adjusted as described with respect to FIGS. 25-28 herein.

The distraction device may also include a fuse like feature or a predetermined failure feature so that the device breaks first before a bone fractures from stresses related to the device implant. This may be accomplished by determining the approximate failure properties of the bones at the location of implant and by designing the distraction rod to fail at a force below the force required to fracture the bone.

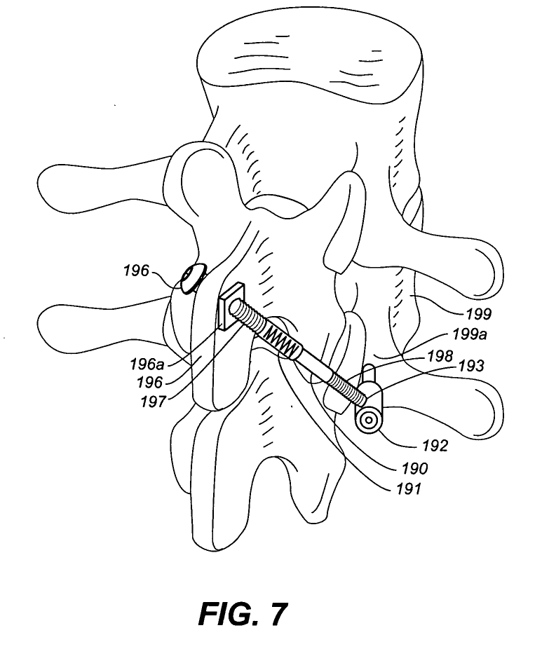

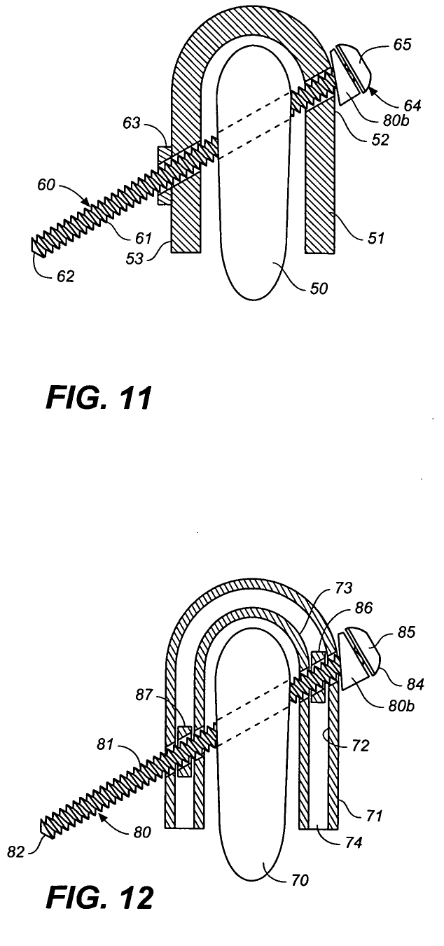

Referring to FIG. 7, a distraction system in accordance with the invention is illustrated where the distraction device is anchored to a pedicle from one level and a spinous process of an adjacent level. In this particular embodiment, the distraction system is positioned from the spinous process of a superior vertebra to the pedicle of a lower or inferior vertebra. The distraction system of an embodiment includes a rod attached or fixed to a spinous process and coupled to a pedicle attachment device that is attached to the pedicle. The pedicle attachment device illustrated in this embodiment comprises a pedicle screw. However, other pedicle anchors or pedicle attachment devices or mechanisms are contemplated herein. The distraction rod 190 may include any of the features of the various distraction rods described herein, for example, the distraction rod may include a distraction element, the distraction rod 190 may adjustable in length in various ways, may be adjustable by different mechanisms including remotely or minimally invasively, and/or the distraction rod 190 may include shock absorbing features or locking features features. The distraction system includes a pedicle screw 192 with a threaded opening 193 for receiving the distraction rod 191. The distraction rod 191 is configured to be anchored to the spinous process 194 of a first vertebra 195 by a rod portion (or screw) 197 extending through the spinous process 194 and having a head 196 holding the rod portion 197 on to the spinous process 194. The a threaded distal end 198 of the rod portion 197 extends into the threaded opening 193 of the pedicle screw 192 which is implanted in the pedicle 199a of a second vertebra 199, and thereby mechanically coupling the first and second vertebrae 195, 199. The distraction rod 190 is implanted so that there is an oblique (i.e., with respect to a median and/or horizontal plane) exertional force between the spinous process 194 of the first vertebra 195 and the pedicle 199a of the second vertebra 199. The distraction rod 190, when in position, operates to exert a separating force in a direction that separates the two vertebrae 195, 199. The distraction rod 190 may be attached to the pedicle screw 192 either before, during or after distraction occurs. An obliquely threaded nut 196a such as nut 80b described with respect to FIG. 11, may tighten screw against the spinous process 194. The spinous process 194 may be reinforced in a manner as described herein. The distraction rod 190 may also be positioned through a posterior arch reinforcing member as described herein. A second distraction rod (not shown) is positioned on the contralateral side of the spinous process 194 and through the contralateral pedicle of the second vertebra 199. The distraction rod 190 is positioned at an oblique angle such that it relieves load from the facet joint between the vertebrae 195, 199. It is believed that relieving the load will decrease pain, slow degeneration of the spine, and reduce formation of osteophytes. Sensors and fracture points may be included with the distraction rod 190 in a similar manner as distraction rod 185 herein.

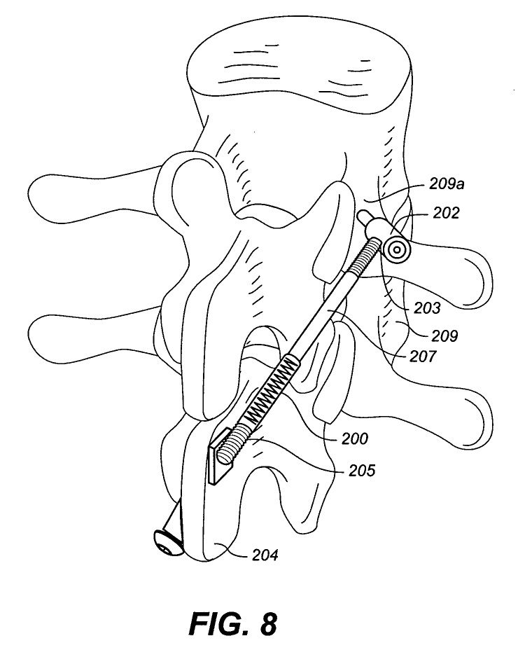

Referring to FIG. 8B, a distraction system in accordance with the invention is illustrated where the distraction device is anchored to a pedicle from one level and a spinous process of an adjacent level. As opposed to the distraction system in FIG. 7, in this particular embodiment, the distraction system is positioned from the spinous process of an inferior or lower vertebra through the pedicle of a superior vertebra. The distraction system of one embodiment includes a rod attached or fixed to a spinous process and coupled to a pedicle attachment device that is attached to the pedicle. The location and angle of the distraction rod may be selected depending on the desired load bearing properties of the distraction system, i.e., depending upon the anatomy the symptoms or prognosis of the patient. The distraction rod 200 may include any of the features of the various distraction rods described herein, for example, the distraction rod 190 may adjustable in length in various ways, may be adjustable by different mechanisms including remote or minimally invasively, and/or the distraction rod 200 may include shock absorbing features or locking features features. The distraction system includes a pedicle screw 202 with a threaded opening 203 for receiving the distraction rod 200. The distraction rod 200 is configured to be anchored to the spinous process 204 of a first vertebra 205 by a rod portion (or screw) 207 extending through the spinous process 204 and having a head 206 holding the rod portion 207 on to the spinous process 204. The a threaded distal end 208 of the rod portion 207 extends into the threaded opening 203 of the pedicle screw 202 which is implanted in the pedicle 209a of a second vertebra 209, and thereby mechanically coupling the first and second vertebrae 205, 209. The distraction rod 200 is implanted so that there is an oblique exertional force between the spinous process 204 of the first vertebra 205 and the pedicle 209a of the second vertebra 209. The spinous process 204 may be reinforced in a manner as described herein. The distraction rod 200 may also be positioned through a posterior arch reinforcing member as described herein. A second distraction rod (not shown) is positioned on the contralateral side of the spinous process 204 and through the contralateral pedicle of the second vertebra 209. The distraction rod 200 is positioned at an oblique angle such that it relieves load from the facet joint between the vertebrae 205, 209. It is believed that relieving the load will decrease pain and reduce formation of osteophytes and increases space for nerves. Sensors and fracture points may be included with the distraction rod 200 in a similar manner as distraction rod 185 herein.

The distraction rods as disclosed herein may also be anchored at oblique angles to different portions of the bony posterior of a vertebra, including but not limited to the lamina, pedicle spinous process and transverse process.

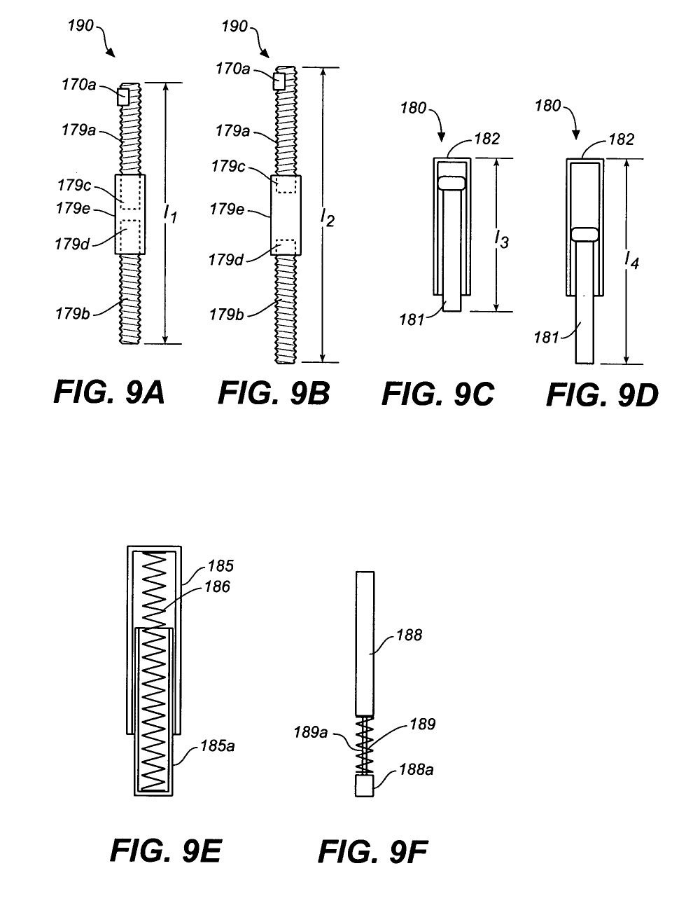

FIGS. 9A and 9B illustrate an enlarged view of the distraction rod 190 of FIG. 7. The distraction rod 190 in which distracting element 179n comprises two opposing rods 179a, 179b with abutting ends 179c 179d and an adjusting device 179e connecting the threaded abutting ends 179c, 179d. In FIG. 9A the ends 179c, 179d of the opposing rods are immediately adjacent each other and the length l₁, of the rod is relatively shorter. In FIG. 5B, the extension by the adjusting device 179e has moved relatively longer. The ends 179c 179d apart from each other and the length 12 of the distraction rod 179 is distraction rod 190 is operable to be extended and locked into an extended position whereby a joint is distracted. The distraction rod 190 may be extendable after implanted to slowly distract the joint until a desired result (e.g., reduction of patient pain or discomfort) is achieved or degree of release of stress on a joint is achieved. This can be visually determined, determined according to patient feedback or determined by a sensor 170a positioned on or adjacent the implanted distraction system 170. (Here it is near the attachment site to the bone.) The sensor 170a may be a strain gauge, an accelerometer, a a piezo-electric film or other sensor that can be used, positioned or configured to determine a mechanical load on the distraction device. The sensor 170a may also be a stand alone sensor positioned in or adjacent a distracted joint and configured to sense a parameter indicative of forces at the joint. The sensor may include an electronic circuit that is configured to telemetrically send a signal containing information correlated to such sensed forces. The electronic circuit may be a passively powered device from an external power source where the external device may interrogate the sensor for information. The electronic circuit may also include signal processing circuits or memory. The distraction rod 190 may include a remotely actuable length adjusting device. For example, the distraction rod 190 may include a mechanical, magnetic or other adjusting device such as a small machine (e.g. a solenoid, a piezoelectric motor or other electromechanical device) that may actuate or move the rod to adjust the degree of distraction. The adjusting device 179e may be actuable by the patient or provider or may automatically adjust, may be adjusted by circuit 179f (that may be telemetrically controlled and/or powered) or may adjust the distraction on demand based at least in part on information sensed by the sensor 170a via control signal through electronic circuit 179f. The distraction rod 190 may also include a predetermined mechanism that is designed to break or fail when a certain force is applied to the device. One or ordinary skill in the art may design the device to release, disengage, fail or break with application of a predetermined or selected force by creating a release mechanism or faults in the material or selecting material or structure specifications. For example the device may be constructed to operate under given normal operating forces but to release, disengage, fail or break prior to a force sufficient to fracture the bone.

FIGS. 9C and 9D illustrate an enlarged view of a variation of a distraction element that may be used with any distraction device or rod described in accordance with the invention. The distraction element 180 comprises opposing rods 181, 182 with rod 181 slidably positioned at least partially within rod 182. The rods 181, 182 longitudinally slide with respect to one another to vary the total length of the distraction element 180. The inner wall of the rod 182 and outer wall of the rod 181 are configured to engage with a detent mechanism, cammed surface or other interference type fit mechanism, when the rods 181, 182 are rotated or actuated or distracted with respect to each other to thereby fix the length of the distraction element 180. FIG. 9C illustrates the distraction element 180 with a relatively shorter length of l₃ and FIG. 9D illustrates the distraction element 180 with a relatively longer length of l₄. The rods 181, 182 may also be simple telescoping tubes that can be crimped or welded or ratcheted together when a desired distraction length is determined.

Referring to FIG. 9E a distraction element 185 that may be used with a distraction device, is illustrated containing a coil or spring-like member 186 where the spring is longitudinally biased so that the coil tends to lengthen, providing a distraction type force. shock absorbing properties. The distraction element 185 may be converted into a rigid or less flexible distraction rod or may be adjusted in flexibility in a manner as described with respect to the devices illustrated in FIGS. 25-28 herein.

Referring to FIG. 9F a distraction element 188 that may be used with a distraction device in accordance with the invention, is illustrated with a spring 189 on one end. The spring 189 is longitudinally biased in a lengthening direction as the spring member 186 described herein with reference to FIG. 9E. The spring 189 is configured to permit movement in a plurality of directions and/or planes. A rubber member 189a is positioned inside the coil and acts to dissipate energy or absorb shock. Thus, the distracting rod 188 provides a distracting force in combination with shock absorbing properties. The rod 188 may also be converted to a rigid distraction rod in a manner described above with reference to the distraction rod 185.

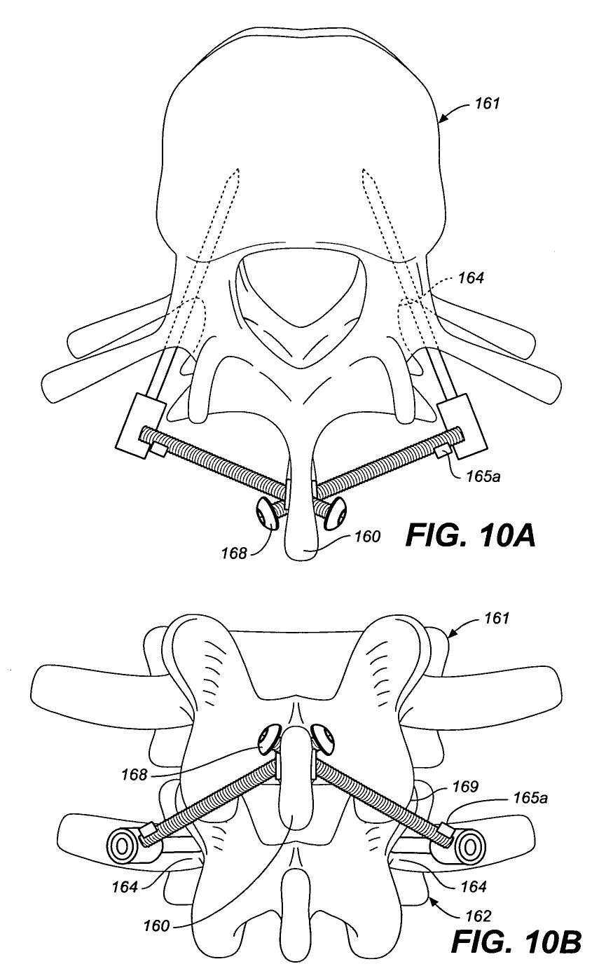

Referring to FIGS. 10A and 10B, a perspective view of the spine is illustrated with a spinal stabilization system in place. A spinous process screw 168 is placed from the contralateral side 165 of the spinous process 160, through the spinous process 160 of a first vertebra 161 and across the facet joint 169 between the first vertebra 161 and an adjacent second vertebra 162, and into the pedicle 164 of the second vertebra 162.

Another feature of the spinous process screw of FIGS. 10A and 10B is that it may be configured to exert flexible, stabilizing, nonfusion forces to the motion segment. For example, this may be used in the event that patient suffers from pain to due laxity of the spinal structures (e.g. degenerative spondylolisthesis). In other words, the looseness of the joint may cause pain. The present invention provides a device and method for dynamically stabilizing (or reducing) such a joint while allowing some flexibility and movement. The device and method provide such stabilization on an oblique angle with respect to the rotational axis of the spine, i.e. at an oblique angle with respect to the median and horizontal planes of the spine. The spinous process and a pedicle are used to anchor a device exerting a stabilizing or compression or contractile force between the two anchors on an oblique angle. Devices that may be used to exert such a contractile force may include, for example, polymeric materials, super elastic metals, and fabrics. The spinous process screw 168 includes a sensor 165a that may be used to sense motion of the distraction device. The forces or stresses on the device may be monitored and used to determine if it is necessary to convert the device to a fusion type device or to otherwise reduce motion. The sensor may also be used as a diagnostic device to measure the amount of joint motion upon insertion of the implant or over time.

The system illustrated in FIGS. 10A and 10B may also be used for the treatment of spondylolysis, to attain stability across the pars interarticularis.

The spinous process may be reinforced in a manner as described herein. The various rods or screws through the spinous process may also be positioned through a posterior arch reinforcing member as described herein.

FIG. 11 illustrates a spinous process rod or screw 60 in accordance with the invention. The spinous process rod or screw 60 comprises an elongate portion 61 configured to extend through the reinforcement hood 51 (for example, as described in further detail herein with reference to FIGS. 3A-4D positioned around spinous process 50 and into an adjacent element such as, e.g. a pedicle screw. The spinous process rod or screw 60 may include threaded portions. The distal end 62 of the rod may be threaded or otherwise configured to engage an adjacent element. The spinous process screw or rod 60 further comprises a proximal securing element 65 located on the proximal portion 64 of the spinous process screw or rod 60. The proximal securing element 65 is configured to engage a first wall 52 portion of the spinous process 60 or reinforcement hood 51. (“Engage” as used herein means to either directly or indirectly engage.) As illustrated, the distal securing element 63 comprises an obliquely threaded nut that is configured to receive screw 61 which is coupled to the hood 51 at an oblique angle with respect to the wall 53. The oblique threaded nut may be used in other applications where a screw is oblique with respect to the abject to which is engaged, coupled or attached. The obliquely threaded nut may have a predetermined angle at which it directs the screw with respec to the hood to guide the desired angle or directions of the screw placement. This may be predetermined base on imaging of a particular patient's anatomy. A distal securing element 63 is provided more distal of the proximal securing element 65. The distal securing element is configured to engage a second wall portion 53 generally opposite the first wall portion 52 so that the spinous process element is secured or fixed to the hood and spinous process. (The term “fix” as used herein means either directly or indirectly fix to and may include dynamic elements.)

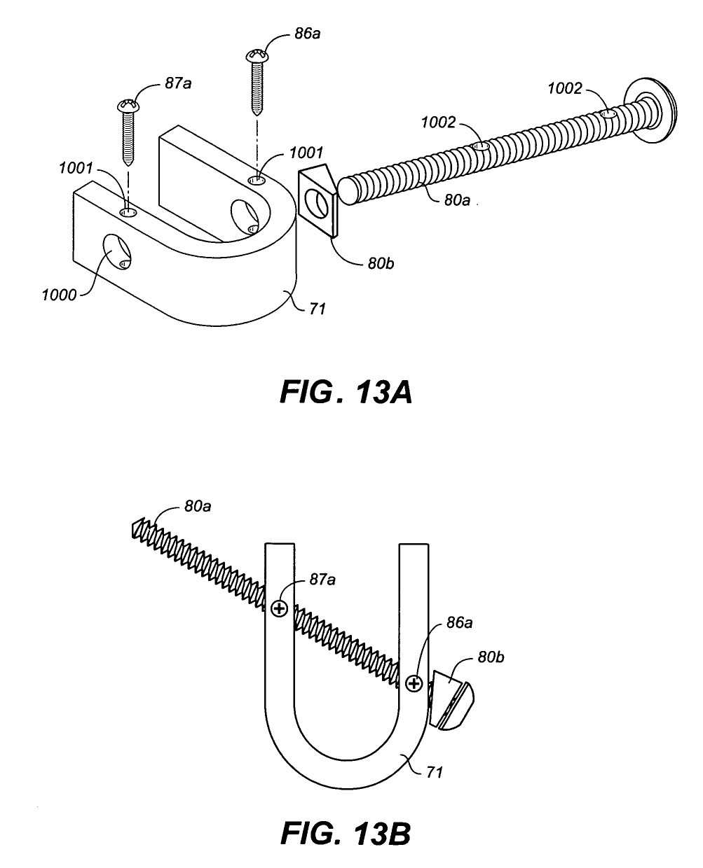

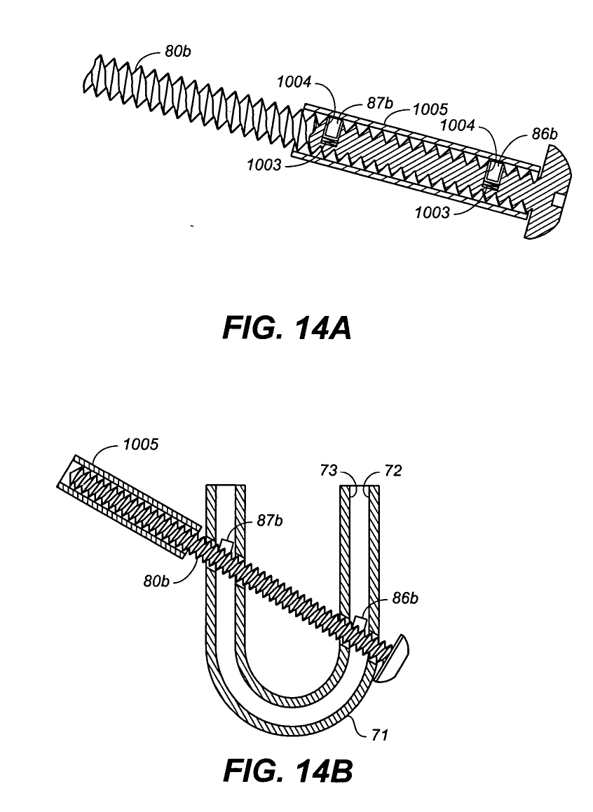

FIG. 12 illustrates a spinous process rod or screw 80 in accordance with the invention. The spinous process rod or screw 80 comprises an elongate portion 81 configured to extend through the reinforcement hood 71 (for example, as described in further detail herein with reference to FIGS. 3A-4D) positioned around spinous process 70 and into an adjacent element such as, e.g. a pedicle screw. The spinous process rod or screw 80 may include threaded portions. The distal end 82 of the rod may be threaded or otherwise configured to engage an adjacent element, e.g. with a connecting member, including but not limited to connecting members described herein. The spinous process screw or rod 80 further comprises a proximal securing element 85 located on the proximal portion 84 of the spinous process screw or rod 80. The proximal securing element 85 is configured to engage a first wall 72 portion of the spinous process 70 or reinforcement hood 71. (“Engage” as is used herein to mean either directly or indirectly engage.) A hollow space or chamber 74 is formed in the reinforcement hood 71 so that the hollow chamber may engageably receive one or more securing elements, e.g. first and second securing elements 86, 87 therein. The securing elements 86, 87 may be positioned on either or both sides of the spinous process 70 through which the screw or rod 80 is positioned. As illustrated in FIG. 12, securing element 86 is positioned on the proximal portion 84 of the screw 80 while securing portion 87 is positioned on the distal portion 82 of the screw 80. Securing elements 86, 87 may be obliquely threaded nuts, for example, as described with respect to nut 80b in FIG. 14A-14B. Securing elements may be attached a variety of ways, for example as illustrated in FIGS. 13A-13B and 14A-14B. FIGS. 13A-13B illustrate manual insertion of securing elements in accordance with the invention. Spinous process screw 80a is placed through both wings of the hood 71 while passing through holes 1000 as shown. Securing elements 86a and 87a are inserted into receiving holes 1001 within the hood 71 and receiving holes 1002 within the spinous process screw 80a. Securing elements 86a, 87a prevent movement of the spinous process screw 80a. FIGS. 14A-14B illustrates automatic deployment of securing elements in accordance with the invention. The securing elements 86b and 87b could be positioned in recesses 1004 in the spinous process screw 80b and spring loaded with springs 1003 attached inside of the recesses 1004. An external sheath 1005 is positioned around the spinous process screw 80b. The screw 80b is positioned through a spinous process and a hood. The securing elements are then deployed upon removal of an external sheath 1005. The securing element 86, 86a, or 86b is configured to engage the first wall portion of the spinous process (or hood) from within the hood 71. The securing element 87, 87a, or 87b is configured to engage a second wall portion 73 generally opposite the first wall portion 72 so that the spinous process element is secured to the hood and spinous process.

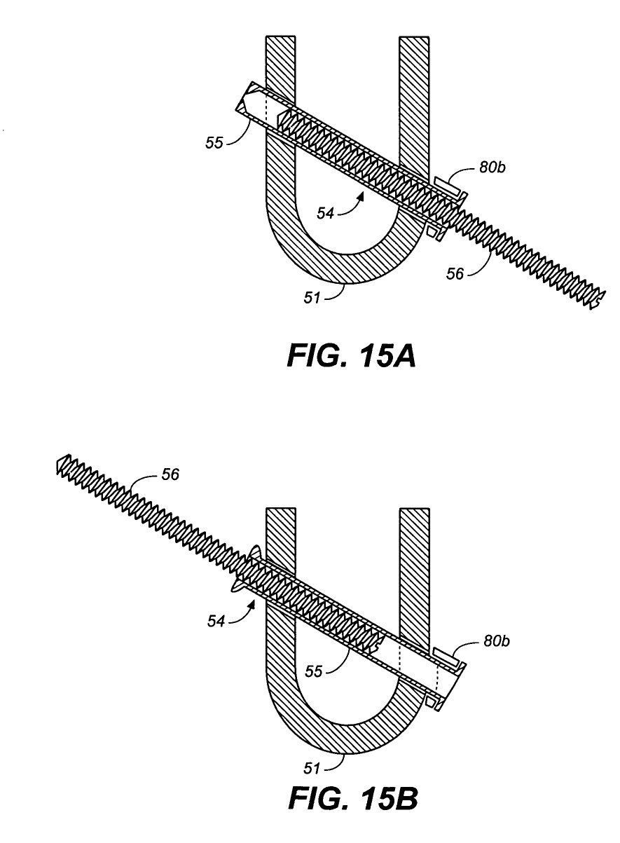

FIGS. 15A and 15B illustrate a spinous device 54, e.g., a process screw or rod, that may be lengthened. This screw or rod 54 may be utilized in any of the distraction devices described herein, to distract the joint across which the spinous process screw or rod is deployed. The screw mechanism may be adjusted over time as well, e.g. with a percutaneously positioned screw driver or the like. The spinous process rod or screw 54 comprises an elongate outer tube portion 55 and an inner rod portion 56. The inner rod portion 56 is configured to move longitudinally within the tube portion 55 to lengthen or shorten the spinous process screw or rod 54. The inner wall of the tube portion 55 may include a threaded inner wall that mates with a threaded outer wall of the rod 54 so that the rod may be screwed to advance the rod 56 and thereby lengthen or shorten the spinous process screw or rod 54. Once the outer rod 55 and screw 56 are positioned within a spinous process or hood 57 the spinous process screw or rod 54 may then be lengthened as shown in FIG. 15B and is configured to extend through the reinforcement hood 51. The lengthened spinous process screw may be used to distract the spinal segment or segments.

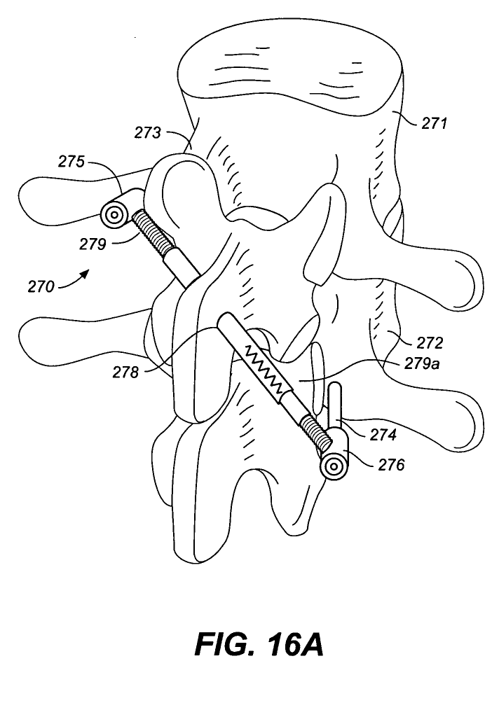

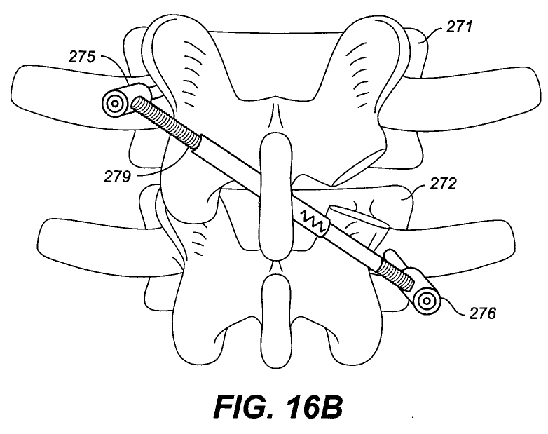

FIGS. 16A and 16B illustrate a support prosthesis configured to provide support of the spine where a facet has been removed in whole or in part. The support prosthesis 270 comprises a support rod 279 anchored into a pedicle 273 of a first vertebra 271 through a screw head of a pedicle screw 275. The support rod 279 extends through an opening 278 in the spinous process 277 to a pedicle screw 276 anchored in contralateral pedicle 274 of a second vertebra 272. The support rod 279 is oriented at an oblique angle with respect to a median and/or horizontal plane intersecting the first vertebra, and over the region 279a from which the facet was removed. The support rod 279 may include a distraction element and/or shock absorbing properties, for example as discussed above with reference to FIGS. 9A-9F. The rod 279 at least in part supports the load that was previously borne by the removed facet joint when it was intact. The support rod 279 also provides distraction for the joint. The spinous process 277 may include reinforcement or a support structure such as described herein. The rod 279 may be constructed of a materiel that permits flexing and twisting motions, such as, e.g., a suitable polymer material. The superior part of the rod 279 may alternatively be anchored in the lamina, spinous process or attachments to the posterior elements of the vertebra. The bar 279 may also be positioned over the region 279a in a generally parallel position with respect to the rotational axis of the spine.

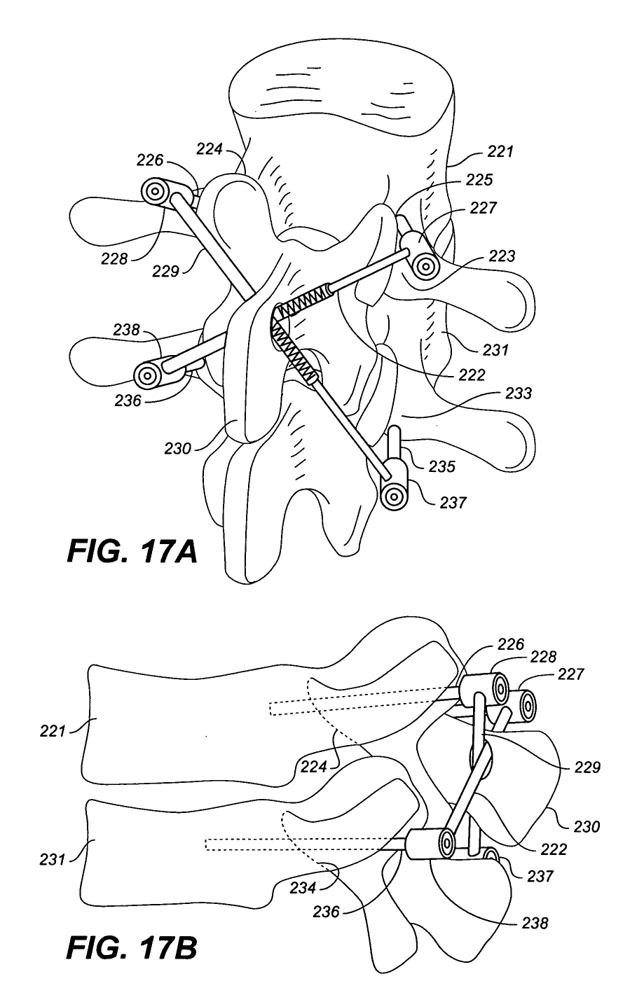

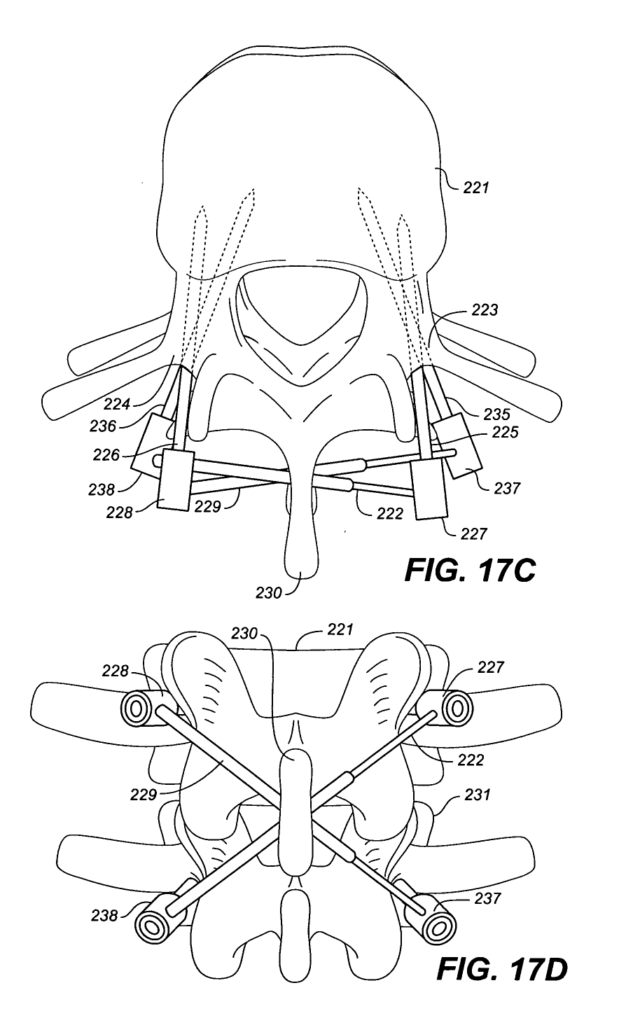

FIGS. 17A-17B illustrate a pedicle to pedicle positioning of a distraction system in accordance with the invention. A pedicle screw 225 is implanted in the pedicle 223 of a first vertebra 221. A pedicle screw 236 is implanted in the pedicle 234 on the contralateral side of a second vertebra 231. A distraction rod 222 is positioned between the pedicle screw 225 on the first vertebra 221 and the pedicle screw 236 on the second vertebra 231 at an oblique angle with respect to the rotational axis along the length of the spine, (or with respect to a median plane and/or a horizontal plane) between the vertebrae 221, 231. The first end of the distraction rod 222 is fixed into a head 227 of the pedicle screw 225 and the opposite end of the distraction rod 222 fixed into a head 238 of the pedicle screw 236. The distraction rod 222 passes through the spinous process 230. The distraction rod 222 includes a distraction element, for example as described above with respect to FIGS. 9A-9E. The spinous process 230 may be reinforced as described herein. Alternatively, the spinous process 230 may be removed to implant the distraction system. A similar distraction rod 229 including a distraction element is affixed on the contralateral pedicles 224, 233 respectively to pedicles 223, 234. A pedicle screw 226 is implanted in the pedicle 224 of a first vertebra 221. A pedicle screw 235 is implanted in the pedicle 233 on the contralateral side of a second vertebra 231. A distraction rod 229 is positioned between the pedicle screw 226 on the first vertebra 221 and the pedicle screw 235 on the second vertebra 231 at an oblique angle with respect to the rotational axis along the length of the spine between the vertebrae 221, 231 (or with respect to a median plane and/or a horizontal plane). The first end of the distraction rod 229 is fixed into a head 228 of the pedicle screw 226 and the opposite end of the distraction rod 229 fixed into a head 237 of the pedicle screw 235. The distraction rod 229 also passes through the spinous process. Or, the spinous process 230 may be removed to implant the distraction system. The distraction rods 222, 229 when in position operate to exert a separating force in a plurality of oblique directions (in this particular instance in opposing directions that are substantially normal with respect to one another, the oblique angle being with respect to a median and/or horizontal plane passing though a vertebra) that separate the two vertebrae 221, 231. The distraction rods 222, 229 may be attached to the pedicle screws 223, 234 and 224, 233 respectively, either before, during or after distraction occurs. Sensors may be included with the distraction rod 222 in a similar manner as distraction rod 185 herein.

The pedicle attachment devices herein may include a sensor that may be used to sensor one or more parameters e.g., strain, pressure, motion, position change, that provides information about possible screw failure. The sensor may communicate the information to an external device, e.g. telemetrically, and may be passively powered by an external device.

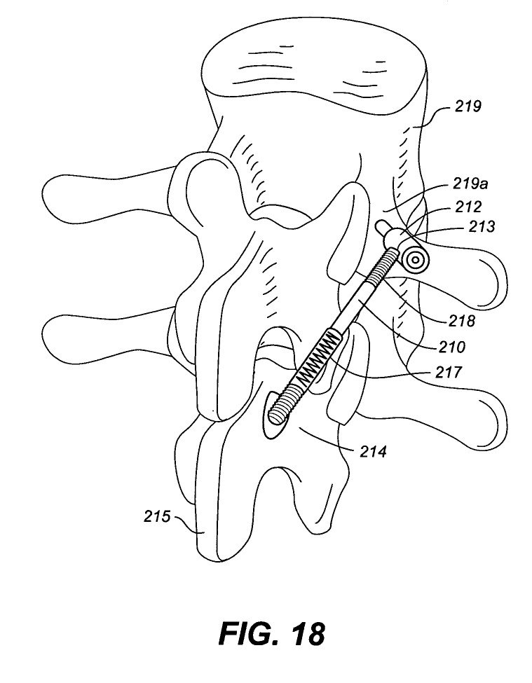

Referring to FIG. 18, a distraction system in accordance with the invention is illustrated where the distraction device is anchored to a pedicle from one level and a lamina of an adjacent level. In this particular embodiment, the distraction system is positioned from the lamina of an inferior or lower vertebra through the pedicle of a superior vertebra. The system may alternatively be positioned form the lamina of a superior vertebra through the pedicle of an inferior vertebra. The location and angle of the distraction rod may be selected depending on the desired load bearing properties of the distraction system, i.e., depending upon the anatomy the symptoms or prognosis of the patient. The distraction rod 210 may include any of the features of the various distraction rods described herein, for example, the distraction rod 210 may adjustable in length in various ways, may be adjustable by different mechanisms including remote or minimally invasively, and/or the distraction rod 210 may include shock absorbing features or locking features. The distraction system includes a pedicle screw 212 with a threaded opening 213 for receiving the distraction rod 210. The distraction rod 210 is configured to be anchored to the lamina 214 of a first vertebra 215 by a rod portion (or screw) 217 extending through the lamina 214 and having a head 216 holding the rod portion 217 on to the lamina 214. The threaded distal end 218 of the rod portion 217 extends into the threaded opening 213 of the pedicle screw 212 which is implanted in the pedicle 219a of a second vertebra 219, and thereby mechanically coupling the first and second vertebrae 215, 219. The distraction rod 210 is implanted so that there is an oblique exertional force between the lamina 214 of the first vertebra 215 and the pedicle 219a of the second vertebra 219. The lamina 214 may be reinforced in a manner as described herein. The distraction rod 210 may accordingly be positioned through a reinforced lamina as described herein. A second distraction rod (not shown) is positioned on the contralateral side of the lamina 214 and through the contralateral pedicle of the second vertebra 219. The distraction rod 210 is positioned at an oblique angle such that it relieves load from the facet joint between the vertebrae 215, 219.

FIG. 19 illustrates a spinal distraction system with a distracting rod 1006 anchored at one end (the cephalic end 1015) to the inferior lip 1007 of a superior vertebra 1008 via a hook 1009, and anchored at the other end (the caudal end) 1014 to hood 1014a configured to secure the rod 1006 to the lamina 1010 of an inferior vertebra 1011.

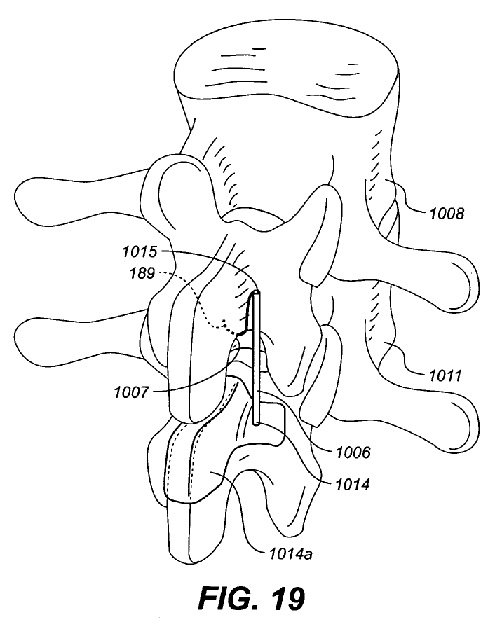

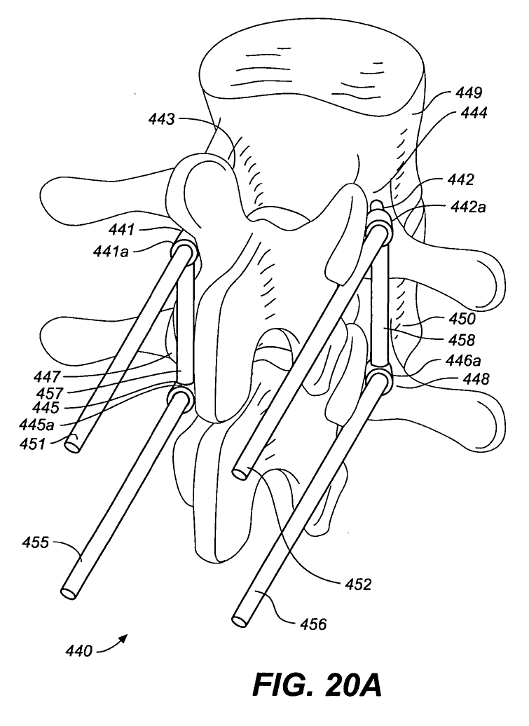

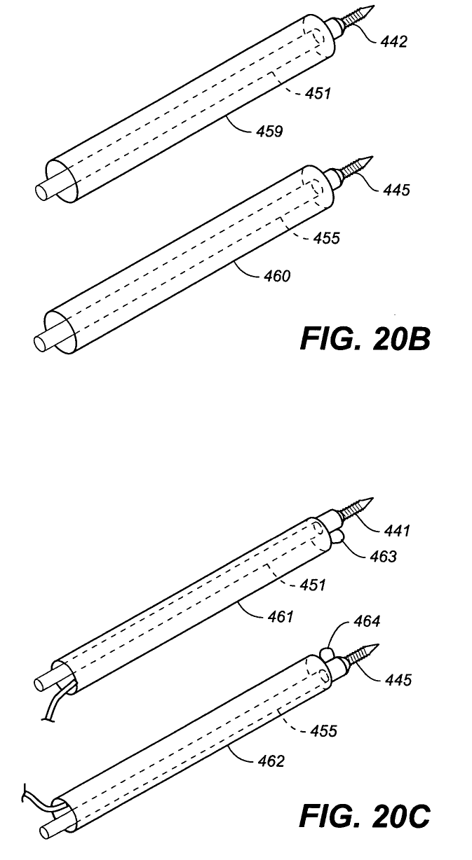

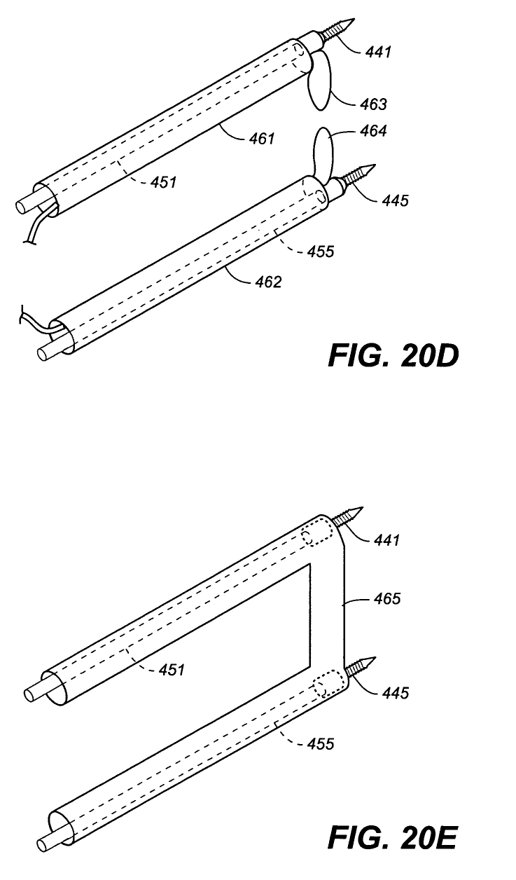

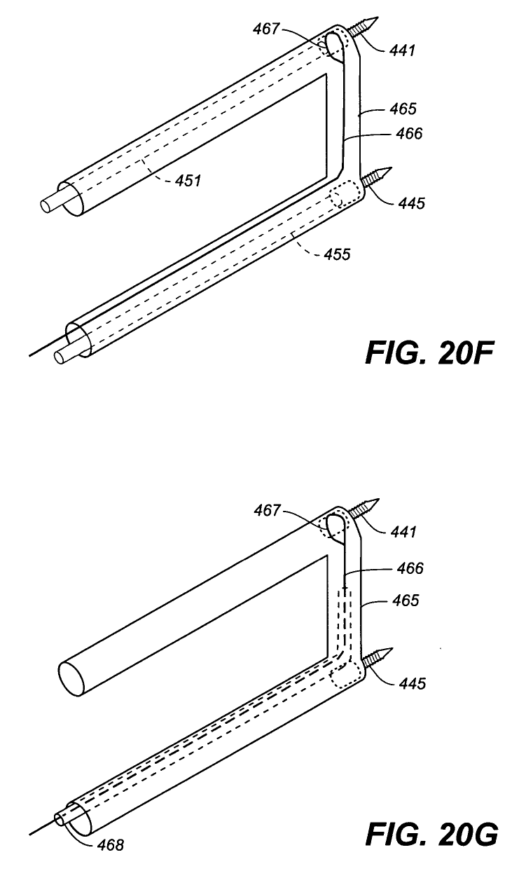

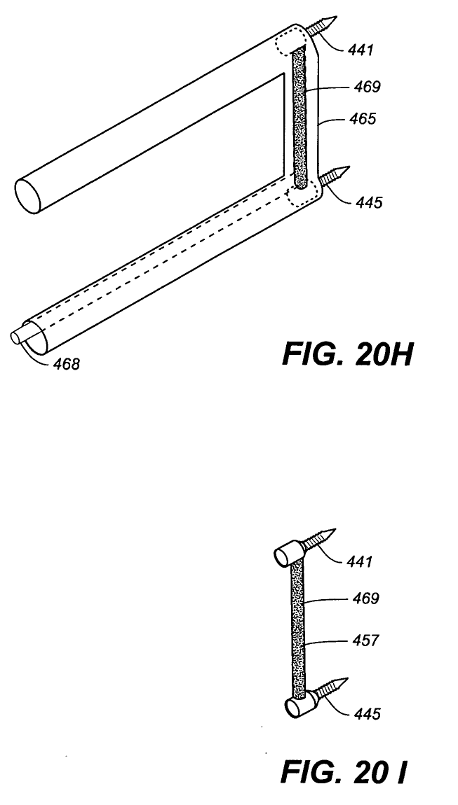

FIGS. 20A-20I illustrate a spinal distraction system 440 and method of implanting in accordance with the invention. The system 440 comprises pedicle screws 441, 442, fixed to contralateral pedicles 443, 444 of a first vertebra 449 and pedicle screws 445, 446 fixed to contralateral pedicles 447, 448 of a second vertebra 450. The system further comprises removable pedicle screw extenders 451, 452, 455, 456 with threaded connector ends. Each of the pedicle screws 441, 442, 445, 446 comprise threaded screw heads 441a, 442a, 445a, 446a configured to receive threaded heads of the pedicle screw extenders 451, 452, 454, 456, respectively. In use, the pedicle screw extenders 451, 452, 455, 456 are coupled to the pedicle screws 441, 442, 445, 446 by way of threaded screw heads 441a, 442a, 445a, 446a. The pedicle screw extenders 451, 452, 455, 456 extend from the pedicle screws 441, 442, 445, 446 at the spine to position just at or outside of the subcutaneous tissue. The pedicle screw extenders 451, and 455, and pedicle screw extenders 452 and 456, are respectively separated from each other to distract the joint motion segments between the first vertebra 449 and the second vertebra 450. This may be done while the patient is awake and standing. The provider may manipulate the screw extenders until the patient reports relief from the pain e.g. of spinal stenosis. Distraction bars 457, 458 are respectively positioned between and coupled to pedicle screw extenders 451, and 455, and pedicle screw extenders 452 and 456 to maintain distraction as described herein with reference to FIG. 20B-20J. The pedicle screw extenders 451, 452, 455, 456 may be unscrewed and removed. A wire may extend from each for the pedicle screws 441, 442, 445, 446 through a lumen in the pedicle screw extenders 451, 452, 455, 456 so that when they are unscrewed and removed, a wire remains in place. If additional adjustment is necessary, the wires may act as guidewires guiding the pedicle screw extenders 451, 452, 455, 456 to the respective pedicles 441, 442, 445, 446 to adjust the distraction level.

Referring to FIGS. 20B-20J a method of placing distraction bars 457, 458 is illustrated. With screw extenders 451, 455 in place, dilators 459, 460 are placed over the screw extenders 451, 455 to create an access channel to the pedicle screws 441, 445. (FIG. 20B) The dilators 459, 460 are then removed and balloon catheters 461, 462 are inserted over the extenders 451, 455. (FIG. 20C) The balloon catheters 461, 462 each have a lumen therethrough for receiving the extenders 451, 455, and inflatable balloons 463, 464 on one side of each of the catheters 461, 462 so that when the balloons are position opposite each other, they may be inflated to form contiguous canal when they meet each other (FIG. 20D). The extenders 451, 455 and balloon catheters 461, 462 may be keyed so that the balloon catheters are appropriately aligned with the balloons 463, 464 facing each other so that a contiguous passageway may be formed. The balloons 463, 464 are deflated and the balloon catheters 461, 462 are removed leaving a tunneled region 465 between the pedicle screws 441, 445. (FIG. 20E).

A guidewire 466 having a wire loop 467 at the end is introduced through the channel adjacent the extender 455 and is directed through the tunneled region 465 where the loop 467 is used to capture the threaded head 441a of the pedicle screw 441. (FIG. 20F) Various imaging techniques such as fluoroscopic imaging may be used to guide the loop 467 to the proper location at the head 441a of the pedicle screw 441. A flexible tube 468 is guided over the guide wire (FIG. 20G) to a position through the tunneled region 465 and to the pedicle screw 441 (FIG. 20H). The guidewire 466 is removed and a curable polymer 469 is injected through flexible tube 468 preferably using a flexible needle that can be positioned at the end of the flexible tube 468 where it sits in the tunneled region 465. (FIG. 20I) The polymer cures and the portion of the tube that is not in the tunneled region is cut away and removed, leaving a hardened tube between the pedicles that holds the pedicle screws 441, 445 in a distracted position with respect to each other. Alternatively, a device such as the Sexant™ device manufactured by Medtronic, Inc. may be used to create a tunnel between adjacent pedicle screws and to connect them with a curved rod.

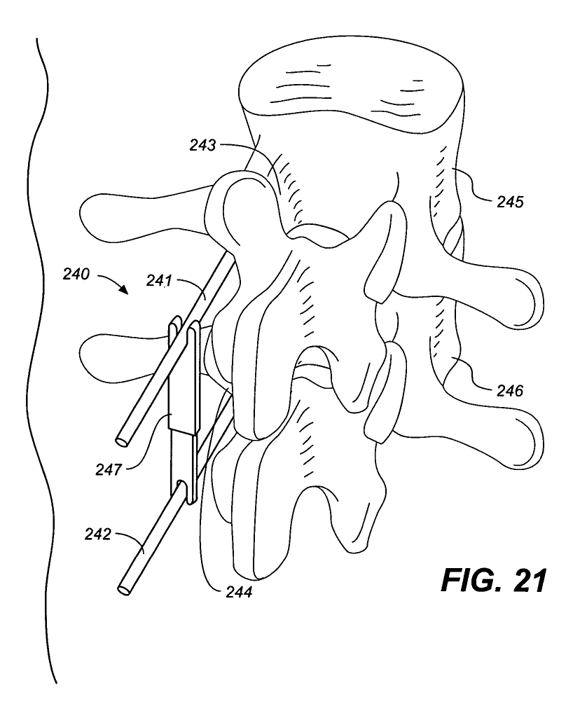

FIG. 21 illustrates an internal fixator for distraction of a motion segment of a spine. The fixator 240 comprises rods 241, 242 placed percutaneously through the skin and muscle to the pedicles 243, 244 of adjacent spinal vertebrae 245, 246 where they are screwed in, or otherwise secured to the pedicles, e.g. via multi-axial pedicle screws. The rods 241, 242 are spread apart to distract the adjacent spinal vertebrae 245, 246 from each other to relieve pressure on the spine and associated tissue at the motion segment between the vertebrae 245, 246. A subcutaneous securing element 247 is placed between the rods 241, 242 in a subcutaneous location between the skin and the muscles, to secure the rods 221, 222 in the distracted position. After positioned, the device may be distracted, e.g. at a physician's office while patient provides feedback to the provider concerning pain or discomfort. This would allow for just enough distraction to relieve symptoms of stenosis, while avoiding unnecessary over-distraction. The securing element 247 may be selected from a plurality of securing elements of different lengths or may itself be distracted. The appropriate length may be selected depending on the amount of distraction of the device. The securing device may replaced at a later time when, for example, further distraction is needed.

According to another aspect of the invention a rod is provided that is anchored to with pedicle screws with screw heads made of or attached to swivel collars, polyaxial heads, or other movable fasteners to allow for near physiologic levels of motion of the spinal motion segment. Angular movement may be provided where a distracting element attaches on either side of a motion segment so that when distracting or lengthening the device, there is accommodation in the device for the change of angle that occurs.

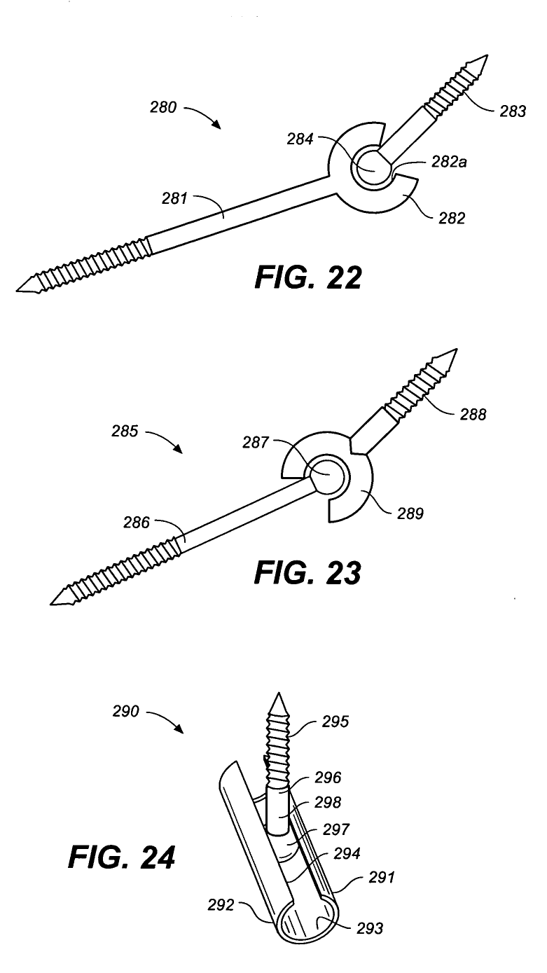

FIG. 22 illustrates an enlarged portion of a spinal prosthesis. The prosthesis 280 may provide support of the load on the spine where a facet has been removed or may provide other support or distraction. The prosthesis 280 comprises a distraction bar 281 used to distract a motion segment of the spine in a number of manners including the distraction devices described herein. A pedicle screw 283 is screwed into a pedicle of the spine or other anatomical location. The distraction bar 281 includes and articulating cup 282 having an inner surface 282a. The pedicle screw 283 has a ball 284 received by and coupled to the cup 282 of the distraction bar 281. In addition to shock absorbing capabilities described in various embodiments herein, the distraction bar 281 also articulates with a portion of the spine to which the pedicle screw 283 is attached.

FIG. 23 illustrates a variation of the prosthesis 280 described with respect to FIG. 22. The prosthesis 285 comprises a distraction bar 286 and an articulating ball 287 configured to engage and couple with an articulation cup 289 of a pedicle screw 288. The prosthesis 285 operates in a similar manner as prosthesis 280.

FIG. 24 illustrates a variation of the prostheses 280, 285 described herein respectively with respect to FIGS. 22 and 23. The prosthesis 290 comprises a distraction bar 291 having an end 292 with a lumen 293 for slidably receiving the end 296 of a pedicle screw 295. The end 296 of the pedicle screw 295 comprises a ball portion 297 attached to a neck 298. The ball portion 297 is configured to slide within the lumen 293 of the distraction bar 291 which contains the ball portion 297. The neck 298 of the pedicle screw 295 extends out of the distraction bar 291 through a longitudinal slit 294 that slidably receives the narrower neck portion 298 of the pedicle screw 295.

One embodiment of the invention is a rod anchored at each end across a motion segment that can be “switched” between dynamic distraction and rigid fixation in a minimally invasive, percutaneous, or non-invasive fashion. One way for this to occur is injection of a flowable material within the lumen of the device, which would cure, and immobilize the components which allow for motion. Electrical current, heat, mechanical energy, or other techniques could also be used to render movable components fixed. Another method is insertion of a rigid implant axially along the length of the dynamic implant. This method of rendering a flexible prosthesis rigid may be applied to the design of other combination motion/fixation prostheses, including disc, facet hip, knee, fingers shoulder, elbows, and ankle prostheses, etc.

FIGS. 25-28 illustrate convertible or adjustable dynamic stabilization devices for joints. The stiffness or flexibility of the device may be altered or titrated after implantation to adapt the stiffness to a particular patient, and/or to adjust the stiffness over time, for example when laxity of the joint increases with age. Referring to FIG. 25 illustrates a dynamic stabilization prosthesis 350. The prosthesis comprises a flexible coil 352 contained in a tube member 351 comprising telescoping tubes. The prosthesis 350 may be used in a number of manners affixed across a joint motion segment to dynamically stabilize the joint. The coil 352 may be energy absorbing. The coil 352 may also be configured to exert a distracting force on the joint when implanted. FIG. 26 illustrates the dynamic stabilization prosthesis 350 of FIG. 25 converted to a rigid or more rigid prosthesis. The prosthesis 350 includes a slit 353 for receiving a rigid wire member 354. In FIG. 26, the rigid wire member 354 is inserted into the slit 353 to form the prosthesis from a dynamic prosthesis into a rigid prosthesis. As an alternative to a rigid wire member, a flexible coil of a selected stiffness may be inserted to change the stiffness of the dynamic prosthesis. The tube may alternatively comprise a ferromagnetic material contained therein and an electromagnetic field is applied that causes the prosthesis to become stiffer. The field may be varied to provide a variety of gradients in stiffness. The device may also include a sensor that operates as sensor 170a described herein. Feedback may be provided and the stiffness of the prosthesis adjusted accordingly. The stiffness may be varied when implanted using patient feedback so that the implant is more or less flexible depending upon an individual patient's needs. In addition the stiffness may be changed at different times during the course of the implants lifetime. For example, the stiffness may be increased when an increased amount of stabilization is required.

FIG. 27 illustrates an alternative prosthesis 360 also comprising a flexible coil 362 contained in a tube member 361. The tube member is configured to receive a fluid material such as a curable polymer 364 that cures in the tubular member to create a rigid prosthesis. As illustrated in FIG. 27 a rigid prosthesis is formed from a dynamic prosthesis by injecting the polymer material 364 into the tubular member 361. The flexibility/stiffness properties of the prosthesis may be selected by selecting such properties of the polymer to be injected.

As illustrated in FIG. 28 a flexible prosthesis 365 is illustrated. The flexibility of the prosthesis 365 is adjustable by injecting a polymer material into one or more of the columnar cavities 367, 368, 369. The polymer may be injected into each cavity at a different time so the stiffness of the prosthesis may be increased gradually over time. The stiffness/flexibility properties of the polymer injected may also be selected according to a desired stiffness/flexibility of the implant.

According to an embodiment of the invention, the dynamic stabilizer may comprise a shock absorber that has both energy absorbing and energy dissipating properties. The tension band effect of the posterior columns may also offload the pressures borne by anterior column of the spine. So in addition to helping to protect the facet joints, other aspects of the invention would help slow the progression of degenerative disc disease, annular degradation, disc herniation, and vertebral compression fractures.

Another aspect of the invention is to supplement implants or repair procedures of the anterior column with a posterior shock absorber device (rod, screw, plate). Examples of these implants or procedures include total disc replacements, annular repair, artificial nucleus, and vertebroplasty/kyphoplasty.

Another aspect of the invention is to supplement implants or repair procedures of the posterior column with a shock absorber rod. Examples of these implants or procedures include interspinous distraction wedges, facet joint replacements, and posterior arch replacements.

Another aspect of the invention provides a posterior support implants with shock absorbing properties, to decrease or remove the load experienced by the facets. Implant components may include springs, coils, hydraulic or fluid filled piston chambers, or elastic materials. Each end of the device could be anchored in such a fashion so the rod bridges the facet joint, reducing the loads borne by the joint. This is believed to reduce wear of the facets and resulting pain and altered spinal biomechanics.

One embodiment of the invention comprises an anchor device with a therapeutic substance or drug delivery device, e.g. a drug port and/or reservoir, or matrix attached to a vertebra. In one embodiment, the device is anchored adjacent a site near where pain is present. The port is configured to deliver steroids or anesthetic agents via a catheter to a desired location, for example, the facet joint, neural foramen, vertebral body, annulus, nucleus, back muscles, back ligaments, bone metastases, intrathecal space, epidural space, or other targets in, on, or around the spine. The catheter can direct the drug to the correct location by positioning the end of the catheter at a target location. The port is configured to be refilled periodically percutaneously, e.g. using an imaging device and a percutaneously placed needle that can inject the refill into the port, e.g. through a biocompatible polymer or rubber type port access mechanism. The device further comprises a patient actuation mechanism for patient control of drug delivery as needed for pain relief, manually or remotely using a telemetrically triggered delivery from an external telemetry control device. According one aspect of the invention such a device is attached to a boney structure of the spine. Other device that may be attached to the spine may include sensory or therapeutic devices, including nerve stimulators, bone growth stimulators and radioactive seeds.

In addition, a structural implant may be anchored to bone, to which a sensory or therapeutic device may be attached. The sensory or therapeutic device could be placed external to the bone, on the surface of the bone, or internal to the bone.

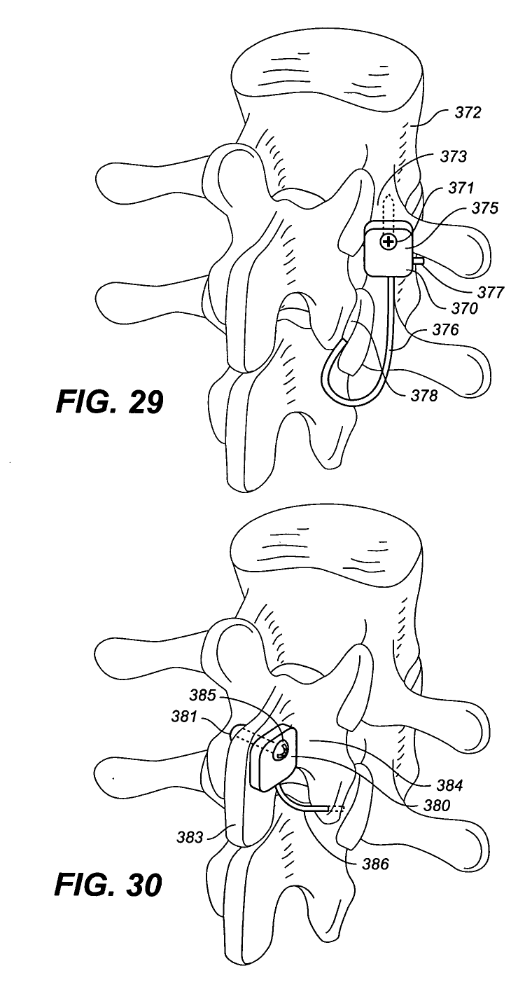

FIGS. 29 and 30 illustrate drug delivery devices 370, 380, respectively, in accordance with the invention. The drug delivery device 370 includes a reservoir 375 attached by an anchor 371 configured to anchor the reservoir 375 to the bone of the spine. In particular, in this embodiment, the anchor 371 comprises a pedicle screw that anchors the device to the pedicle 373 of a vertebra 372. The reservoir 375 includes a catheter 376 in communication with the contents of the reservoir 375 and having an end positioned adjacent or in a zygapophyseal joint 378 where the drug is directed to have a therapeutic effect on the joint 378. The device may include a telemetrically actuable pump mechanism for delivering the drug to the joint upon telemetric actuation by an external control device. The device 370 further comprises a port 377 for receiving (e.g. via a percutaneously introduced needle) into the reservoir 375, refills of the therapeutic substance or drug. Device 380 comprises a similar catheter 386, and reservoir 385 attached by an anchor 381 to the spinous process 383 or alternatively an adjacent lamina 384. The spinous process 383 or lamina 384 may be reinforced prior to attachment of the anchor 381 or may be attached to a reinforcement device positioned at the posterior arch of the spine, as described herein with reference to FIGS. 1A-4D.

1. A spine distractor comprising:
an elongate member configured to be positioned across at least one motion segment between vertebrae of the spine wherein the elongate portion defines an oblique angle with respect to a median plane of a patient and a horizontal plane extending through said first vertebra:
a first portion coupled to the elongate member and configured to couple to a spinous process of a first vertebra of a spine;
a second portion configured to be coupled to a bony portion of a second vertebra, wherein the elongate portion is located between the first portion and the second portion; and
a distraction element located between the first portion and the second portion, wherein the distraction element is configured to exert a distracting force to the motion segment through the elongate member.

2. The spine distractor of claim 1 wherein the distraction element comprises a spring biased in a distracting direction.

3. The spine distractor of claim 2 wherein the distraction element comprises a coil.

4. The spine distractor of claim 1 wherein the distraction element comprises an expandable portion.

5. The spine distractor of claim 1 wherein the distraction element comprises a shock absorbing element.

6. The spine distractor of claim 5 further comprising a spring in parallel with said shock absorbing element.

7. The spine distractor of claim 1 wherein the first portion comprises an anchor portion configured to couple the first portion to the spinal process.

8. The spine distractor of claim 7 wherein the anchor portion comprises a head portion.

9. The spine distractor of claim 1 wherein the second portion is configured to couple to a pedicle attachment device attached to a pedicle of the second vertebra.

10. The spine distractor of claim 9 wherein the pedicle has an adjustable length.

11. The spine distractor of claim 9 wherein the second portion comprises a first articulating surface and the pedicle attachment device comprises a second articulating surface, and wherein one of the first articulating surface and second articulating surface is configured to articulate with respect to the other of the first articulating surface and the second articulating surface.

12. The spine distractor of claim 1 further comprising a dynamic element configured to permit limited movement of the distractor when implanted.

13. The spine distractor of claim 12 wherein the elongate member has a length and wherein the dynamic element is configured to permit limited movement of the elongate member along said length.

14. A spine implant comprising:
an elongate portion configured to be positioned across a joint of a spine,
a proximal portion configured to be fixed to a first bone portion of a first vertebra, the proximal portion located on a first side with respect to the elongate portion and a distal portion configured to be coupled to a second bone portion of a second vertebra, the distal portion located on a second side with respect to the elongate portion, wherein the spine implant has a length, and wherein length is adjustable when the implant is attached to the first bone portion and the second bone portion.

15. The spine implant of claim 14 wherein the elongate portion is adjustable in length.

16. The spine implant of claim 14 wherein the length is remotely adjustable

17. The spine implant of claim 14 further comprising an adjustment element configured to lengthen or shorten the implant wherein the adjustment element, when implanted, is percutaneously actuable.

18. The spine implant of claim 14 wherein the elongate portion comprises a distraction rod configured to exert a distraction force across the at least one joint.

19. The spine implant of claim 14 further comprising a moveable portion wherein the length changes when the moveable portion moves.

20. The spine implant of claim 19 wherein the moveable portion comprises a spring.

21. The spine implant of claim 19 further comprising a fixing element configured to fix the length of the implant after the moveable portion moves to change the length.
